(19) United States
(12) Patent Application Publication
(10) Pub. No.: US 2022/0226449 A1
(43) Pub. Date: Jul. 21, 2022

(54) IMMUNOGENIC COMPOSITIONS

(71) Applicant: Telethon Kids Institute, Nedlands, Western Australia (AU)

(72) Inventor: Jason Waithman, Nedlands, Western Australia (AU)

(73) Assignee: Telethon Kids Institute, Nedlands, Western Australia (AU)

(21) Appl. No.: 17/614,786

(22) PCT Filed: May 29, 2020
(86) PCT No.: PCT/AU2020/050541

§ 371 Date: Nov. 29, 2021

(30) Foreign Application Priority Data
May 31, 2019 (AU) 2019901876

(51) Int. Cl. A61K 39/00; A61K 38/21; A61K 39/395; A61P 35/00

(52) U.S. Cl. CPC A61K 39/0011; A61K 38/215; A61K 2039/5152; A61P 35/00; A61K 39/3955

(57) ABSTRACT

The present invention provides an immunogenic composition comprising, consisting essentially of or consisting of: a) a cell expressing an interferon-β (IFN-β) receptor agonist, and b) a tumour antigen. The invention also provides methods of treatment using said compositions.

### FIELD OF THE INVENTION

The present invention relates to compositions and modified cancer cells for use in the prevention and/or treatment of cancer. The invention also relates to methods and uses of those compositions and modified cancer cells in the prevention and/or treatment of cancer.

### RELATED APPLICATION

This application claims priority from Australian provisional application AU 2019901876, the entire contents of which are hereby incorporated by reference.

### BACKGROUND OF THE INVENTION

In spite of numerous advances in medical research, cancer remains a leading cause of death throughout the developed world. Non-specific approaches to cancer management, such as surgery, radiotherapy and generalized chemotherapy, have been successful in the management of a selective group of circulating and slow-growing solid cancers. However, many solid tumours are considerably resistant to such approaches, and the prognosis in such cases is correspondingly grave.

One example is brain cancer. Each year, approximately 15,000 cases of high grade astrocytomas are diagnosed in the United States. The number is growing in both paediatric and adult populations. Standard treatments include cytoreductive surgery followed by radiation therapy or chemotherapy. There is no cure, and virtually all patients ultimately succumb to recurrent or progressive disease. The overall survival for grade IV astrocytomas (glioblastoma multiforme) is poor, with 50% of patients dying in the first year after diagnosis.

A second example is ovarian carcinoma. This cancer is the fourth most frequent cause of female cancer death in the United States. Because of its insidious onset and progression, 65 to 75 percent of patients present with tumour disseminated throughout the peritoneal cavity. Although many of these patients initially respond to the standard combination of surgery and cytotoxic chemotherapy, nearly 90 percent develop recurrence and inevitably succumb to their disease.

Because these tumours are aggressive and highly resistant to standard treatments, new therapies are needed.

An emerging area of cancer treatment is immunotherapy. The general principle is to confer upon the subject being treated an ability to mount what is in effect a rejection response, specifically against the malignant cells. There are a number of immunological strategies under development, including: 1. Adoptive immunotherapy using stimulated autologous cells of various kinds; 2. Systemic transfer of allogeneic lymphocytes; 3. Intra-tumour implantation of immunologically reactive cells; and 4. Vaccination at a distant site to generate a systemic tumour-specific immune response.

Most cancer cells elicit an immune response that is evident by the presence of immune cell infiltrates and inflammation. This response, however, is not strong enough to overcome the cancer cell's defence strategies. The lack of understanding of the complex interactions between tumours and the immune system has hindered the development of cancer immunotherapy. Approaches involving using purified tumour antigens and more complex mixtures of tumour antigens have often failed to stimulate adequate immune responses against tumours. The reasons for this are unknown, but may include the genetic instability of tumours and the ability of tumours to evade the immune system by presenting a “normal” appearance or releasing inhibitors. Tumours can respond to an immune response by reducing the amount of targeted antigens, by masking antigens from the immune system or by expressing mutated versions of antigens that are no longer recognised. Such defensive strategies undermine the immune system, making it difficult to maintain an effective immune response at the level required to halt tumour growth and cause regression. Moreover, the responses may be inadequate since they fail to stimulate an adaptive immune response.

There has been a lack of success in the development of vaccines that generate effective cellular immune responses for the treatment or prevention of cancer. A similar lack of success has also applied to infectious diseases. Consequently, there is a need for new or improved compositions that stimulate the immune system for the treatment or prevention of cancer or an infectious disease.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

In one aspect, the present invention provides an immunogenic composition comprising, consisting essentially of or consisting of:

a) a cell expressing an interferon-β (IFN-β) receptor agonist, and

b) a tumour antigen.

The IFN-β receptor agonist produced by a cell defined in any aspect of this invention may be secreted from the cells, or present on the outer membrane of the cells. Where the IFN-β receptor agonist has a local immunostimulatory effect, it can be preferable that it be primarily attached to the cell membrane to keep it in the vicinity of bystander tumour antigen comprised in the cell. Where the IFN-β receptor agonist has a recruitment effect, it can be preferable that it be primarily secreted. As a third option, the IFN-β receptor agonist can be synthesized by the cell in both membrane-associated and secreted forms.

In any embodiment of the invention, the tumour antigen in the immunogenic composition is provided in or on a cell. Accordingly, the invention provides an immunogenic composition comprising, consisting essentially of or consisting of:

a) a cell expressing an interferon-β (IFN-β) receptor agonist, and

b) a cell expressing a tumour antigen.

The cell expressing the tumour antigen may be a modified human cancer cell. For example, the cell may be a human cancer cell that has been inactivated, or irradiated to prevent the cell from developing a tumour.

In alternative embodiments of the invention, the composition comprises, consists of or consists essentially of a cell that has been genetically modified to express the tumour antigen and the IFN-β receptor agonist. Accordingly, the present invention provides an immunogenic composition comprising, consisting essentially of or consisting of a cell expressing an IFN-β receptor agonist and expressing a tumour antigen.

The tumour antigen is any antigenic substance produced by tumour cells and can trigger an immune response in the host. Typically, the tumour antigen is a protein, or a fragment, peptide or derivative thereof, produced by a tumour cell. Preferably, the tumour antigen is a tumour-specific antigen. The tumour antigen may be an antigen from any cancer. The tumour antigen may be provided in the form of a lysate from a human cancer cell. The lysate may be derived from a cancer cell obtained from the individual requiring treatment. Alternatively, the lysate may be derived from a pool of individuals having the same or different cancers.

The cell expressing an IFN-β receptor agonist may be a cytotoxic lymphocyte, such as a T cell or a Natural Killer (NK cell). Alternatively, the cell expressing an IFN-β receptor agonist may be a professional antigen presenting cell, preferably a dendritic cell (DC), macrophage or a B cell.

Accordingly, in a further aspect, the present invention provides an immunogenic composition comprising, consisting essentially of or consisting of: a) a T cell, NK cell or professional antigen presenting cell expressing an IFN-β receptor agonist, and b) a tumour antigen.

In certain embodiments, the T cell, NK cell or professional antigen presenting cell expressing the IFN-β receptor agonist also expresses the tumour antigen. Accordingly, in a further aspect, the present invention provides an immunogenic composition comprising, consisting essentially of or consisting of a T cell, NK cell or professional antigen presenting cell expressing an IFNβ receptor agonist and expressing a tumour antigen.

The T cells may be selected from the group consisting of tumour infiltrating lymphocytes, peripheral blood lymphocyte, γδ T cells, enriched with mixed lymphocyte tumour cell cultures (MLTCs) or cloned using autologous antigen presenting cells and tumour derived peptides. The lymphocytes may be isolated from a histocompatible donor, or from the cancer-bearing subject.

In any method of the invention, the antigen presenting cells, NK cells or T cells are purified or substantially purified prior to culture. This step enriches the APCs, NK cells or T cells by removing other cell types from the biological sample.

The APCs, NK cells or T cells may be a population that includes more than one type of APC or T cell, comprising any one or more types described herein. For example, the population of T cells may include naïve, activated and/or memory T cells.

In a further aspect of the invention, the cell in the immunogenic composition may be a human cancer cell wherein the tumour antigen is an antigen expressed by the human cancer cell. In this context, the cancer cell may be modified to express the IFN-β receptor agonist.

Accordingly, the invention also provides an immunogenic composition comprising, consisting essentially of or consisting of a modified human cancer cell expressing an IFN-β receptor agonist.

In any aspect of the invention, the IFN-β receptor agonist may be the IFN-β polypeptide, or an immunologically active fragment, variant or derivative thereof, including those described herein. In alternative aspects of the invention, the IFN-β receptor agonist may be an IFN-β mimetic, including any of those described herein.

Still further, the IFN-β receptor agonist may be an antibody that binds to the IFN-β receptor and agonises the receptor.

In another aspect, the invention provides a pharmaceutical composition for treating or preventing cancer in an individual comprising, consisting essentially of or consisting of:

a) a cell expressing an interferon-β (IFN-β) receptor agonist,

b) a tumour antigen,

and a pharmaceutically acceptable diluent, excipient or carrier.

In another aspect, the invention provides a pharmaceutical composition for treating or preventing cancer in an individual comprising, consisting essentially of or consisting of as an active ingredient:

a) a cell expressing an interferon-β (IFN-β) receptor agonist,

b) a tumour antigen,

and a pharmaceutically acceptable diluent, excipient or carrier.

In another aspect, the invention provides a pharmaceutical composition for treating or preventing cancer in an individual comprising, consisting essentially of or consisting of as a main ingredient:

a) a cell expressing an interferon-β (IFN-β) receptor agonist,

b) a tumour antigen,

and a pharmaceutically acceptable diluent, excipient or carrier.

In any embodiment of the invention, the pharmaceutical composition comprises, consists essentially of or consists of a cell expressing an interferon-β (IFN-β) receptor agonist and expressing a tumour antigen. In certain embodiments, the cell is a modified human cancer cell expressing an IFN-β receptor agonist. Alternatively, the cell may be a T cell, NK cell or antigen presenting cell which expresses a tumour antigen and an IFN-β receptor agonist.

Still further, the pharmaceutical composition may comprise, consist essentially of, or consist of a cell expressing an IFN-β receptor agonist, and a cell expressing a tumour antigen, such as a modified human cancer cell that has been modified to prevent the cell from forming a tumour.

The present invention also provides a method of treating cancer comprising administering an immunogenic composition, modified cancer cell or pharmaceutical composition of the invention as described herein to an individual in need thereof, thereby treating the cancer.

The present invention also provides a method of treating cancer comprising
providing an individual in need of cancer treatment; and
administering an immunogenic composition, modified cancer cell or pharmaceutical composition of the invention as described herein to the individual,
thereby treating the cancer.

In any embodiment, the method of treating comprising administering an immunogenic composition, modified cancer cell or pharmaceutical composition of the invention as described herein, further includes administration of additional therapies. For example, the method preferably also includes administration of a checkpoint inhibitor. Examples of suitable checkpoint inhibitors include therapies that bind to and inhibit CTLA4, PD-1, and/or PD-L1, including but not limited to: Atezolizumab, Spartalizumab, Pembrolizumab, Nivolumab, and Ipilimumab.

In another aspect, the present invention also provides a use of an immunogenic composition, modified cancer cell of the invention as described herein in the manufacture of a medicament for the treatment or prevention of cancer in an individual.

In another aspect, the present invention also provides an immunogenic composition, modified cancer cell or pharmaceutical composition of the invention as described herein for use in the treatment or prevention of cancer in an individual.

In any aspect, the cancer may be any cancer described herein.

In any aspect, the individual may be any individual as described herein.

The present invention also provides a method for inducing an immune response suitable for the treatment of cancer in an individual, the method comprising administering an immunogenic composition, modified cancer cell or pharmaceutical composition of the invention as described herein to an individual in need thereof, thereby treating the cancer.

The present invention also provides a method of inducing a T cell immune response in an individual, the method comprising administering an immunogenic composition, modified cancer cell or pharmaceutical composition of the invention as described herein to an individual in need thereof, thereby inducing a T cell immune response. Preferably the T cell immune response is a cytotoxic T cell immune response.

The present invention also provides a method of treating cancer in an individual, the method comprising
obtaining a cancer cell from an individual in need of treatment for cancer,
modifying the cancer cell to (a) reduce or remove the ability of the cell to form a tumour and (b) express an IFN-β receptor agonist, and
administering the modified cancer cell to the individual,

thereby treating cancer in the individual.

The present invention also provides a method for producing a modified cancer cell, the method comprising
providing a cancer cell,
inactivating the cancer cell to reduce or remove the ability of the cell to form a tumour, and
modifying the inactivated cancer cell to express an IFNβ receptor agonist, thereby producing a modified cancer cell.

In any of the above methods, the IFN-β receptor agonist may be an IFN-β polypeptide, or an immunologically active fragment, variant or derivative thereof, including those described herein. In alternative embodiments of the above methods, the IFN-β receptor agonist may be an IFN-β mimetic, including any of those described herein.

In any aspect of the invention, modifying the cancer cell to express an interferon beta receptor agonist may be by modifying the genome of the cancer cell to increase expression of interferon beta receptor agonist, or introduction of an exogenous nucleic acid encoding an interferon beta receptor agonist.

In certain embodiments, the cancer cell for use in any composition or method described herein, may comprise an autologous cancer cell from the subject being treated. In other embodiments, the cancer cell comprises an allogenic cancer cell. Those of skill in the art are familiar with methods for obtaining cancer cells from a subject. For example, the cancer cell composition may be obtained by biopsy, aspiration, surgical resection, venipuncture, or leukapheresis. In certain aspects, the cancer cell is expanded in culture prior to modification (such as by transfection).

Although tumour antigens from cancer cells, and cancer cell of any cancer type are contemplated by the present invention, particular examples of cancer cells include breast cancer cells, lung cancer cells, prostate cancer cells, ovarian cancer cells, brain cancer cells, liver cancer cells, cervical cancer cells, colon cancer cells, renal cancer cells, skin cancer cells, including melanoma cells, head & neck cancer cells, bone cancer cells, esophageal cancer cells, bladder cancer cells, uterine cancer cells, lymphatic cancer cells, stomach cancer cells, pancreatic cancer cells, testicular cancer cells, or leukemia cells.

In any embodiment of the invention, the individual requiring treatment is a mammal. Preferably, the mammal is a human. In certain embodiments, the individual has a hyperproliferative disease, such as cancer. In other embodiments, the individual is at risk for developing cancer.

In any aspect of the invention, modifying the cancer cell to express an interferon beta receptor agonist may be by modifying the genome of the cancer cell to increase expression of interferon beta receptor agonist, or introduction of an exogenous nucleic acid encoding an interferon beta receptor agonist.

In any embodiment of the invention, the interferon beta receptor agonist may be IFN-β or a variant or fragment thereof.

The immunogenic composition, modified cancer cell or pharmaceutical composition of the present invention can be administered to a subject by methods well known to those of skill in the art. For example, the composition or cell may be administered by intravenous injection, intramuscular injection, intratumoural injection, or subcutaneous injection. It is also contemplated that the composition or cell may be administered intranodally, intralymphaticly, or intraperitoneally. The composition or cell may be administered to the subject at or near a tumour in the subject, or to a site from which a tumour has been surgically removed from the subject. However, it is not necessary that the composition or cell be administered at the tumour site to achieve a therapeutic effect. Thus, in certain embodiments the composition or modified cancer cells may be administered at a site distant from the tumour site. Those of skill in the art will be able to determine the best method for administering the composition or modified cancer cells to an individual.

It is desirable to inactivate a cancer cell prior to administration to the subject. Those of skill in the art are familiar with methods for inactivating cells. In some embodiments, the cancer cell is inactivated by a cytostatic agent or a cytotoxic agent. In other embodiments, a cancer cell is inactivated by irradiation. In another embodiment, the cancer cell is co-transfected with a suicide gene, such as HSV-TK.

A cancer cell transfected with HSV-TK could then be killed after it was administered to the subject by giving the subject ganciclovir. A combination of cell inactivating methods may also be used.

The immunogenic composition or modified cancer cell of the invention may be administered to the subject as a vaccine. The vaccine may be used therapeutically or prophylactically. A therapeutic vaccine is administered to a subject having cancer to treat the cancer. In a subject having cancer, the vaccine may be made from the subject's own cancer cells. However, allogenic cancer cells could also be used. A prophylactic vaccine is administered to a subject without cancer to reduce the risk of the subject developing cancer.

As used herein, except where the context requires otherwise, the term “comprise” and variations of the term, such as “comprising”, “comprises” and “comprised”, are not intended to exclude further additives, components, integers or steps.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1: schematic of the experimental design to measure tumour-specific CD8+ T cell expansion post vaccination. Tracking of this T cell response is made possible by generating B16 melanoma cell lines that express the model antigen glycoprotein B (gB) derived from herpes simplex virus. This B16.gB line was used to generate multiple independent lines engineered to express a single type I interferon, with B16.gB.IFN-β an example. T cell responses were tracked to the model antigen gB following injection of mice with a low precursor frequency of congenic gB-specific naïve TCR transqenic CD8+ T cells prior to anti-tumour vaccination with irradiated recombinant B16 cells, with expansion of the tumour-specific transgenic CD8+ T cells measured seven days post vaccination.

FIG. 2: IFN-β significantly enhances CD8+ T cell expansion.

FIG. 3: Tumour-specific CD8+ T cell expansion is lost in IFNAR^{o/o} mice.

FIG. 4: IFN-β significantly expands the endogenous tumour-specific CD8+ T cell compartment.

FIG. 5: Endogenous tumour-specific CD8+ T cell expansion is lost in IFNAR^{o/o} mice.

FIG. 6: Endogenous CD8+ T cell expansion is reduced in I/AE^{o/o} mice and CD4+ help is required for optimal expansion.

FIG. 7: Schematic of the experimental prophylactic vaccination protocol in a mouse model of subcutaneous melanoma. At Day 0, mice receive an i.p. vaccination of irradiated B16.gB.IFN cells. At Day 7, mice are challenged with a subcutaneous inoculation of B16.gB cells

FIG. 8: Percentage of tumour free survival in C57BL/6 mice up to 100 days after prophylactic vaccination.

FIG. 9: Percentage of tumour free survival in IFNAR^{o/o} mice up to 100 days after prophylactic vaccination.

FIG. 10: Percentage of tumour free survival in I/AE^{o/o} mice up to 100 days after prophylactic vaccination.

FIG. 11: Schematic of the experimental therapeutic vaccination protocol in a mouse model of cutaneous melanoma. At Day 0, mice are challenged with an epicutaneous graft of B16.gB cells. At Day 4, the mice receive an i.p. vaccination of irradiated B16.gB.IFN cells.

FIG. 12: Therapeutic vaccination with IFN-β enhances tumour-free survival and long-term protection FIG. 12A shows tumour-free survival in C57BL/6 mice up to 60 days after therapeutic vaccination. FIG. 12B shows percentage of cured mice that either remained tumour-free survival or developed palpable tumours after B16.gB re-challenge subcutaneously in opposite flank.

FIG. 13: Cross-presenting XCR1+ DCs are essential for CD8+ T cell expansion. Transgenic XCR1-DTR mice express a primate diphtheria toxin (DTx) receptor (DTR), allowing for conditional depletion of cross-presenting XCR1+ DCs in the presence of DTx. The expansion of tumour-specific CD8+ T cells was measured in vaccinated XCR1-DTR mice treated with either saline (PBS) or DTx.

FIG. 14: Vaccination with IFNβ synergises with anti-PDL1 checkpoint blockade therapy to delay tumour progression. Overall survival of mice challenged with 5×10⁵ B16.gB cells subcutaneously. Mice received (A) vaccination alone with 2.5×10⁶ irradiated B16.Kbloss.gB.GFP±IFNα1 OR IFNβ (n=10 per group) i.p. three days post-tumour inoculation or (B) vaccination plus three doses of 200 μg anti-PDL1 (n=10 per group) i.p. on days 6, 9 and 12 post-tumour inoculation. Statistical significance was determined by Log-rank Mantel-Cox test where *p<0.1, **p<0.05, ****p<0.001.

FIG. 15: Vaccination with IFNβ synergises with anti-PDL1 checkpoint blockade therapy to delay tumour progression. Individual tumour growth of mice challenged with 5×10⁵ B16.gB cells subcutaneously. Mice received the indicated vaccination (A) alone or (B) in combination with three doses of anti-PDL1 (n=10 per group).

### DETAILED DESCRIPTION OF THE EMBODIMENTS

It will be understood that the invention disclosed and defined in this specification extends to all alternative combinations of two or more of the individual features mentioned or evident from the text or drawings. All of these different combinations constitute various alternative aspects of the invention.

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims.

One skilled in the art will recognize many methods and materials similar or equivalent to those described herein, which could be used in the practice of the present invention. The present invention is in no way limited to the methods and materials described. It will be understood that the invention disclosed and defined in this specification extends to all alternative combinations of two or more of the individual features mentioned or evident from the text or drawings. All of these different combinations constitute various alternative aspects of the invention.

All of the patents and publications referred to herein are incorporated by reference in their entirety.

Definitions

Unless specifically indicated otherwise, all technical and scientific terms used herein have the same meaning as commonly understood by those of ordinary skill in the art to which this invention belongs. In addition, any method or material similar or equivalent to a method or material described herein can be used in the practice of the present invention. For purposes of the present invention, the following terms are defined.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth. For purposes of interpreting this specification, terms used in the singular will also include the plural and vice versa.

The terms “about” and “approximately” shall generally mean an acceptable degree of error for the quantity measured given the nature or precision of the measurements. Typical, exemplary degrees of error are within 20 percent (%), preferably within 10%, and more preferably within 5% of a given value or range of values. Alternatively, and particularly in biological systems, the terms “about” and “approximately” may mean values that are within an order of magnitude, preferably within 5-fold and more preferably within 2-fold of a given value. Numerical quantities given herein are approximate unless stated otherwise, meaning that the term “about” or “approximately” can be inferred when not expressly stated.

The terms “subject,” “individual,” and “patient” are used interchangeably herein to refer to a vertebrate, preferably a mammal, more preferably a human. Mammals include, but are not limited to, murines, rats, simians, humans, farm animals, sport animals, and pets. Tissues, cells and their progeny of a biological entity obtained in vivo or cultured in vitro are also encompassed.

As used herein, the term “administering” includes oral administration, topical contact, administration as a suppository, intravenous, intraperitoneal, intramuscular, intralesional, intratumoural, intradermal, intralymphatic, intrathecal, intranasal, or subcutaneous administration to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc.

The term “treating” refers to an approach for obtaining beneficial or desired results including, but not limited to, a therapeutic benefit and/or a prophylactic benefit. By therapeutic benefit is meant any therapeutically relevant improvement in or effect on one or more diseases, conditions, or symptoms under treatment. Therapeutic benefit can also mean to effect a cure of one or more diseases, conditions, or symptoms under treatment.

The term “effective amount” or “sufficient amount” refers to the amount of a modified cancer cell or other composition that is sufficient to effect beneficial or desired results. The therapeutically effective amount may vary depending upon one or more of:

the subject and disease condition being treated, the weight and age of the subject, the severity of the disease condition, the manner of administration and the like, which can readily be determined by one of ordinary skill in the art. The specific amount may vary depending on one or more of: the particular agent chosen, the target cell type, the location of the target cell in the subject, the dosing regimen to be followed, whether it is administered in combination with other compounds, timing of administration, and the physical delivery system in which it is carried.

For the purposes herein an effective amount is determined by such considerations as may be known in the art. The amount must be effective to achieve the desired therapeutic effect in a subject suffering from cancer. The desired therapeutic effect may include, for example, amelioration of undesired symptoms associated with cancer, prevention of the manifestation of such symptoms before they occur, slowing down the progression of symptoms associated with cancer, slowing down or limiting any irreversible damage caused by cancer, lessening the severity of or curing a cancer, or improving the survival rate or providing more rapid recovery from a cancer.

The effective amount depends, inter alia, on the type and severity of the disease to be treated and the treatment regime. The effective amount is typically determined in appropriately designed clinical trials (dose range studies) and the person versed in the art will know how to properly conduct such trials in order to determine the effective amount. As generally known, an effective amount depends on a variety of factors including the distribution profile of a therapeutic agent (e.g., a whole-cell cancer vaccine) or composition within the body, the relationship between a variety of pharmacological parameters (e.g., half-life in the body) and undesired side effects, and other factors such as age and gender, etc.

The term “pharmaceutically acceptable carrier” refers to a substance that aids the administration of an active agent to a cell, an organism, or a subject. “Pharmaceutically acceptable carrier” refers to a carrier or excipient that can be included in the compositions of the invention and that causes no significant adverse toxicological effect on the subject. Non-limiting examples of pharmaceutically acceptable carriers include water, NaCl, normal saline solutions, lactated Ringer's, normal sucrose, normal glucose, binders, fillers, disintegrants, lubricants, coatings, sweeteners, flavors and colors, liposomes, dispersion media, microcapsules, cationic lipid carriers, isotonic and absorption delaying agents, and the like. The carrier may also be substances for providing the formulation with stability, sterility and isotonicity (e.g. antimicrobial preservatives, antioxidants, chelating agents and buffers), for preventing the action of microorganisms (e.g. antimicrobial and antifungal agents, such as parabens, chlorobutanol, sorbic acid and the like) or for providing the formulation with an edible flavor etc. In some instances, the carrier is an agent that facilitates the delivery of a modified cancer cell to a target cell or tissue. One of skill in the art will recognize that other pharmaceutical carriers are useful in the present invention.

Interferon Beta Receptor Agonist

The interferon-α/β receptor (IFNAR) is a heteromeric cell surface receptor composed of two subunits, referred to as the low affinity subunit, IFNAR1, and the high affinity subunit, IFNAR2. Upon binding of type I interferons, IFNAR activates the JAK-STAT signalling pathway, along with MAPK, PI3K, and Akt signaling pathways. Type I IFN receptor forms a ternary complex, composed of its two subunits IFNAR1 and IFNAR2, and a type I IFN ligand. Ligand binding to either subunit is required for and precedes dimerization and activation of the receptor. Each subunit of IFNAR contains an N-terminal ligand binding domain (with two or four fibronectin type II-like subdomains, for IFNAR2 and IFNAR1, respectively), a transmembrane (TM) domain, and a cytoplasmic domain. Each type I IFN ligand contains a “hotspot”, or a sequence of conserved amino acids that are involved in binding to the receptor, specifically the high affinity receptor IFNAR2, which determines the affinity of each ligand for the receptor.

As used herein, an “interferon (IFN)-β receptor agonist” means a molecule that binds to IFN-alpha/beta receptor (IFNAR), subunits IFNAR-1 or IFNAR-2, and which elicits a response typical of IFN-β. An exemplary response includes any one or more of the functions of the IFNAR, particularly those described herein. Typically the IFN-β receptor agonist comprises, consists essentially of or consists of a polypeptide.

As used herein, a fragment of interferon beta is preferably a fragment that binds to and activates an interferon beta receptor. Typically, the fragment of interferon beta binds to and activates the same receptors as full length interferon beta.

Preferably, the fragment of the interferon beta binds to IFNAR present on the surface of a cell, preferably an immune cell, resulting in phosphorylation of one or more tyrosine residues on an IFNAR.

Type I IFNs bind to IFNAR1 or IFNAR2, forming a binary complex. The binary complex further recruits the remaining IFNAR subunit, completing the ternary complex and activating downstream JAK/STAT signaling. IFN ligation to IFNAR brings the receptor associated kinases, JAK1 and Tyk2, into close proximity, resulting in kinase transphosphorylation and subsequent phosphorylation of tyrosines on IFNAR1 and IFNAR2. Phosphotyrosine residues on IFNAR1 and IFNAR2 recruit STAT proteins (classically STAT1, STAT2, or STAT3, although STAT4, STAT5, and STAT6 may play a role in certain cell types) via their SH2 domains. Once recruited, STAT proteins are phosphorylated by which induces their homo- or heterodimerization. These dimers translocate to the nucleus, binding interferon-stimulated response elements (ISRE) and gamma activating sequences (GAS), promoting gene transcription.

Non-limiting examples of IFN-β receptor agonists include, for example, the IFN-β polypeptide. Mammalian IFN-β sequences such as human (Gray and Goeddel (1982). Nature, 298:859); rat (Yokoyama, et al., (1997). Biochem Biophys Res Commun., 232:698); canine (Iwata, et al., (1996). J Interferon Cytokine Res., 10:765); porcine (J Interferon Res., (1992).12:153) are known in the art. Another example of IFN-β receptor agonist is an IFN-β receptor agonist antibody (eg anti-IFN anti-idotypic antibody (Osheroff et al. (1985). J Immunol, 135:306).

Non-limiting examples of IFN-β receptor agonist antibodies include mammalian, human, humanized or primatized forms of heavy or light chain, VH and VL, respectively, immunoglobulin (Ig) molecules. “Antibody” refers to any monoclonal or polyclonal immunoglobulin molecule, such as IgM, IgG, IgA, IgE, IgD, and any subclass thereof. The term “antibody” also includes functional fragment of immunoglobulins, such as Fab, Fab′, (Fab′)2, Fv, Fd, scFv and sdFv, unless otherwise expressly stated.

The term “IFN-β receptor antibody” means an antibody that specifically binds to IFN-β receptor (IFNAR). Specific binding is that which is selective for an epitope present in IFN-β receptor. Selective binding can be distinguished from non-selective binding using assays known in the art (e.g., immunoprecipitation, ELISA, Western blotting).

The term “human” when used in reference to an antibody, means that the amino acid sequence of the antibody is fully human, i.e., human heavy and light chain variable and constant regions. All of the antibody amino acids are coded for in the human DNA antibody sequences or exist in a human antibody. An antibody that is non-human may be made fully human by substituting the non-human amino acid residues with amino acid residues that exist in a human antibody.

Amino acid residues present in human antibodies, CDR region maps and human antibody consensus residues are known in the art (see, e.g., Kabat, Sequences of Proteins of Immunological Interest, 4th Ed.US Department of Health and Human Services. Public Health Service (1987); Chothia and Lesk (1987). J. Mol. Biol. 186:651; Padlan (1994). Mol. Immunol. 31:169; and Padlan (1991). Mol. Immunol. 28:489). Methods of producing human antibodies are known in the art (see, for example, WO 02/43478 and WO 02/092812).

The term “humanized” when used in reference to an antibody, means that the amino acid sequence of the antibody has non-human amino acid residues (e.g., mouse, rat, goat, rabbit, etc.) of one or more determining regions (CDRs) that specifically bind to the desired antigen in an acceptor human immunoglobulin molecule, and one or more human amino acid residues in the Fv framework region (FR), which are amino acid residues that flank the CDRs. Human framework region residues of the immunoglobulin can be replaced with corresponding non-human residues. Residues in the human framework regions can therefore be substituted with a corresponding residue from the non-human CDR donor antibody. A humanized antibody may include residues, which are found neither in the human antibody nor in the donor CDR or framework sequences. Methods of producing humanized antibodies are known in the art (see, for example, U.S. Pat. No. 5,225,539; 5,530,101, 5,565,332 and 5,585,089; Riechmann, et al., (1988). Nature 332:323; EP 239,400; W091/09967; EP 592,106; EP 519,596; Padlan (1991). Molecular Immunol. 28:489; Studnicka et al., (1994). Protein Engineering 7:805; and Roguska. et al., (1994). Proc. Nat'l. Acad. Sci. USA 91:969).

Antibodies referred to as “primatized” in the art are within the meaning of “humanized” as used herein, except that the acceptor human immunoglobulin molecule and framework region amino acid residues may be any primate residue, in addition to any human residue.

The invention also includes the use of IFN-β peptides and mimetics, IFN-β receptor agonist peptides and mimetics, and modified (variant) forms, provided that the modified form retains, at least partial activity or function of unmodified or reference peptide or mimetic. For example, a modified IFN-β peptide or mimetic will retain at least a part of IFN-β receptor activating activity. Modified (variant) peptides can have one or more amino acid residues substituted with another residue, added to the sequence or deleted from the sequence. Specific examples include one or more amino acid substitutions, additions or deletions (e.g., 1-3, 3-5, 5-10, 10-20, or more). A modified (variant) peptide can have a sequence with 50%, 60%, 70%, 75%, 80%, 85%, 90%, 95%, 96%, 97%, 98%, or more identity to a reference sequence (e.g., IFN-β). The crystal structure of recombinant interferon-beta (IFN-β) can also be employed to predict the effect of IFN-β modifications (Senda, et al., (1992). EMBO J. 11:3193).

As used herein, the terms “mimetic” and “mimic” refer to a synthetic chemical compound which has substantially the same structural and/or functional characteristics as the reference molecule. The mimetic can be entirely composed of synthetic, non-natural amino acid analogues, or can be a chimeric molecule including one or more natural peptide amino acids and one or more non-natural amino acid analogs. The mimetic can also incorporate any number of natural amino acid conservative substitutions as long as such substitutions do not destroy activity. As with polypeptides which are conservative variants, routine testing can be used to determine whether a mimetic has detectable IFN-β receptor activating activity.

Peptide mimetic compositions can contain any combination of non-natural structural components, which are typically from three structural groups: a) residue linkage groups other than the natural amide bond (“peptide bond”) linkages; b) non-natural residues in place of naturally occurring amino acid residues; or c) residues which induce secondary structural mimicry, i.e., induce or stabilize a secondary structure, e.g., a beta turn, gamma turn, beta sheet, alpha helix conformation, and the like. For example, a polypeptide can be characterized as a mimetic when one or more of the residues are joined by chemical means other than an amide bond. Individual peptidomimetic residues can be joined by amide bonds, non-natural and non-amide chemical bonds other chemical bonds or coupling means including, for example, glutaraldehyde, N-hydroxysuccinimide esters, bifunctional maleimides, N,N′-dicyclohexylcarbodiimide (DCC) or N,N′-diisopropylcarbodiimide (DIC). Linking groups alternative to the amide bond include, for example, ketomethylene (e.g., —C(═O)—CH2— for —C(═O)—NH—), aminomethylene (CH2—NH), ethylene, olefin (CH═CH), ether (CH2—O), thioether (CH2-S), tetrazole (CN4—), thiazole, retroamide, thioamide, or ester (see, e.g., Spatola (1983) in Chemistry and Biochemistry of Amino Acids, Peptides and Proteins, Vol. 7, pp 267-357, “Peptide and Backbone Modifications,” Marcel Decker, NY).

A “conservative substitution” is the replacement of one amino acid by a biologically, chemically or structurally similar residue. Biologically similar means that the substitution is compatible with biological activity. Structurally similar means that the amino acids have side chains with similar length, such as alanine, glycine and serine, or having similar size. Chemical similarity means that the residues have the same charge or are both hydrophilic or hydrophobic. Particular examples include the substitution of one hydrophobic residue, such as isoleucine, valine, leucine or methionine for another, or the substitution of one polar residue for another, such as the substitution of arginine for lysine, glutamic for aspartic acids, or glutamine for asparagine, serine for threonine, and the like.

A specific example of an IFN-β variant is Betaseron, an analogue of human beta-interferon in which serine is substituted for cysteine at position 17. A specific example of an IFN-β mimetic is SYR6 (Sato and Sone, (2003). Biochem J., 371(Pt 2):603). Modified IFN-β sequence candidates are described, for example, in U.S. Pat. No. 6,514,729-recombinant interferon-beta muteins; U.S. Pat. No. 4,793,995-modified (1-56) beta interferons; U.S. Pat. No. 4,753,795-modified (80-113) beta interferons; and U.S. Pat. No. 4,738,845-modified (115-145) beta interferons. It will be understood that the present invention is not limited to those IFN-β variants referenced above but includes any IFN-β variant and any IFN-β receptor agonist.

Peptides and peptidomimetics can be produced and isolated using any method known in the art. Peptides can be synthesized, whole or in part, using chemical methods known in the art (see, e.g., Caruthers (1980). Nucleic Acids Res. Symp. Ser. 215; Horn (1980). Nucleic Acids Res. Symp. Ser. 225; and Banga, A.K., Therapeutic Peptides and Proteins, Formulation, Processing and Delivery Systems (1995) Technomic Publishing Co., Lancaster, Pa.). Peptide synthesis can be performed using various solid-phase techniques (see, e.g., Roberge (1995) Science 269:202; Merrifield (1997). Methods Enzymol. 289:3) and automated synthesis may be achieved, e.g., using the ABI 431 A Peptide Synthesizer (Perkin Elmer) in accordance with the manufacturer's instructions.

Antigens

An “antigen” according to the invention covers any substance that will elicit an immune response. In particular, an “antigen” relates to any substance, preferably a peptide or protein, that reacts specifically with antibodies or T-lymphocytes (T cells). According to the present invention, the term “antigen” comprises any molecule which comprises at least one epitope. Preferably, an antigen in the context of the present invention is a molecule which, optionally after processing, induces an immune reaction, which is preferably specific for the antigen (including cells expressing the antigen). According to the present invention, any suitable antigen may be used, which is a candidate for an immune reaction, wherein the immune reaction is preferably a cellular immune reaction. In the context of the embodiments of the present invention, the antigen is preferably presented by a cell, preferably by an antigen presenting cell which includes a diseased cell, in particular a cancer cell, in the context of MHC/HLA molecules, which results in an immune reaction against the antigen. An antigen is preferably a product which corresponds to or is derived from a naturally occurring antigen. Such naturally occurring antigens include tumour antigens.

Preferably, the antigen peptides according to the invention are MHC class I and/or class II presented peptides or can be processed to produce MHC class I and/or class II presented peptides. Preferably, the antigen peptides comprise an amino acid sequence substantially corresponding to the amino acid sequence of a fragment of an antigen.

In any aspect of the invention, the antigen comprises a peptide capable of being presented by an HLA class I molecule. Preferably, the antigen also comprises a peptide capable of being presented by an HLA class II molecule. Typically, the peptide capable of being presented by an HLA class I molecule comprises a CD8+ T cell epitope. Typically, the peptide capable of being presented by an HLA class II molecule comprises a CD4+ T cell epitope. The antigen typically comprises a HLA-I-restricted T cell epitope and a HLA-II-restricted T cell epitope. Preferably the antigen comprises a CD4+ T cell epitope and a CD8+ T cell epitope that result in activation and/or proliferation of a CD4+ T cell and a CD8+ T cell respectively. Exemplary antigens are any tumour associated antigens, including those described herein.

In any aspect of the invention, the antigen results in an immune response being raised against cells characterized by expression of the antigen and preferably by presentation of the antigen such as diseased cells, in particular cancer cells.

In a preferred embodiment, the antigen is a tumour antigen, i.e., a part of a tumour cell such as a protein or peptide expressed in a tumour cell which may be derived from the cytoplasm, the cell surface or the cell nucleus, in particular those which primarily occur intracellularly or as surface antigens of tumour cells. According to the present invention, a tumour antigen preferably comprises any antigen which is expressed in and optionally characteristic with respect to type and/or expression level for tumours or cancers as well as for tumour or cancer cells. In one embodiment, the term “tumour antigen” or “tumour-associated antigen” relates to proteins that are under normal conditions specifically expressed in a limited number of tissues and/or organs or in specific developmental stages, for example, the tumour antigen may be under normal conditions specifically expressed in stomach tissue, preferably in the gastric mucosa, in reproductive organs, e.g., in testis, in trophoblastic tissue, e.g., in placenta, or in germ line cells, and are expressed or aberrantly expressed in one or more tumour or cancer tissues. In this context, “a limited number” preferably means not more than 3, more preferably not more than 2. The tumour antigens in the context of the present invention include, for example, differentiation antigens, preferably cell type specific differentiation antigens, i.e., proteins that are under normal conditions specifically expressed in a certain cell type at a certain differentiation stage, cancer/testis antigens, i.e., proteins that are under normal conditions specifically expressed in testis and sometimes in placenta, and germ line specific antigens. Preferably, the tumour antigen or the aberrant expression of the tumour antigen identifies cancer cells. In the context of the present invention, the tumour antigen that is expressed by a cancer cell in a subject, e.g., a patient suffering from a cancer disease, is preferably a self-protein in said subject. In preferred embodiments, the tumour antigen in the context of the present invention is expressed under normal conditions specifically in a tissue or organ that is non-essential, i.e., tissues or organs which when damaged by the immune system do not lead to death of the subject, or in organs or structures of the body which are not or only hardly accessible by the immune system.

According to the invention, the terms “tumour antigen”, “tumour expressed antigen”, “tumour associated antigen”, “cancer antigen” and “cancer expressed antigen” are equivalents and are used interchangeably herein. Those skilled in the art will also appreciate that the terms “tumour antigen”, “tumour expressed antigen”, “cancer antigen” and “cancer expressed antigen” can include multiple tumour epitopes or antigens encoded within the same contiguous sequence.

Exemplary antigens include those: (a) comprising the minimal HLA-class-I restricted T cell epitopes described in Sachin et al. Nature (2017) 547:222-226, preferably in the Extended Data Table 3; (b) comprising the class I epitopes described in Ott et al. Nature (2017) 547: 217-221; (c) comprising the peptide neoantigens described in Creaney et al. (2015) Oncoimmunology. 4(7):e1011492; (d) comprising the neoantigens described in McGranaha et al. Science (2016) 351(6280):1463-9; comprising the neoantigens described in Rizvi et al. (2015) 351(6280):1463-9.

Examples of antigens associated with tumours include, but are not limited to: am11, adenomatous polyposis coli protein (APC), Annexin I, Annexin II, adenosine deaminase-binding protein (ADAbp), BAGE, carboxyanhydrase-IX (CAIX), Carcinoembryonic Antigen (CEA), Carcinoembryonic Antigen (CEA) epitope CAP-1, Carcinoembryonic Antigen (CEA) epitope CAP-2, CD133 antigen (also known as prominin 1), Colorectal associated antigen (CRC)-0017-1A/GA733, Ab2 BR3E4, CI17-IA/GA733, cTAGE-1, Cytochrome oxidase 1, E-cadherin, α-catenin, β-catenin and γ-catenin, NeuGcGM3, Cyclophilin B, RCASI, cdc27, CDK4, Dipeptidyl peptidase IV (DPPIV), etv6, disialoganglioside (GD2), epidermal growth factor receptor (EGFR), epithelial cell adhesion molecule (EpCAM), Erythropoietin-producing hepatocellular receptor A2 (EphA2), ErbB-2/neu, c-erbB-2, EP-CAM/ICSA, α-fetoprotein, fibroblast activation protein α (FAP), folate receptor alpha (FR-α), fodrin, Fos related antigen, FucosylGM1, glypican-3 (GPC3),GA733/EoCam, GAGE-1,2, gp100/pmel 17, gp96-associated cellular peptide, G250, Glycolipid antigen-GM2, GD2 or GD3, GM3, Glycoprotein (mucin) antigens-Tn, GnT-V, GTPase activating protein, Hsp70, Hsp90, Hsp96, Hsp105, Hsp110, HSPPC-96, stress protein gp96 (a human colorectal cancer tumour rejection antigen), hepatocyte growth factor receptor (also known as tyrosine-protein kinase Met, hepatocyte growth factor receptor, HGFR or cMET), HP59, human epidermal growth factor receptor-2 (HER2, HER2/neu), human telomerase reverse transcriptase (hTERT), Interleukin 13 receptor alpha 2 (IL13Rα2), L1cell adhesion molecule (L1-CAM), LAGE-1, L19H1, MAZ, Mammaglobin, Melan-A/MART-1, mesothelin (MSLN),an MITF, MITF-A, MITF-M, melanoma GP75, MBTAA, msa, Mucin-1 (MUC-1), CA125 (MUC-16), a MAGE family antigen, a MUC family antigen, NY-BR-1, NY-BR-2 NY-BR-3, NY-BR-4 NY-BR-5, NY-BR-6 NY-BR-7, NY-ESO-1, p120ctn, PARIS-1, PGP 9.5, PRAME, Prostate Specific Antigen (PSA), PSA epitope PSA-1, PSA epitope PSA-2, PSA epitope PSA-3, Ad5-PSA, prostate stem-cell antigen (PSCA), prostate-specific membrane antigen (PSMA), Prostatic Acid Phosphatase (PAP), Prostate epithelium-derived Ets transcription factor (PDEF), Parathyroid-hormone-related protein (PTH-rP), PLU1, Oncofetal antigen-immature laminin receptor (OFA-iLR), PINCH, PRAME, Prp1p/Zer1p, PHF3, Ran, RAGE, a Rab-GAP (Rab GTPase-activating) protein, p21ras, RCAS 1, receptor tyrosine kinase-like orphan receptor 1 (ROR1), SART3, SCP-1, a Smad tumour antigen, SSX-1, SSX-2, SSX-4, STn, sp100 SCP-1, Sialyl-Tn (STn), thyroglobulin, TRP-1, TRP-2, tyrosinase, TF, S2, Telomerase rt peptide, TAG-72, TRAG-3, vascular endothelial growth factor receptor (VEGFR), Wilms tumour gene (WT1), or an immunogenic fragment thereof.

The tumour antigen may be in the form of a polypeptide corresponding to a tumour protein, or may be a peptide fragment, or derivative of a tumour protein. If a peptide is to be presented directly, i.e., without processing, in particular without cleavage, it has a length which is suitable for binding to an HLA molecule, in particular a class I HLA molecule, and preferably is 7-20 amino acids in length, more preferably 7-12 amino acids in length, more preferably 8-11 amino acids in length, in particular 9 or 10 amino acids in length.

If a peptide is part of a larger entity comprising additional sequences and is to be presented following processing, in particular following cleavage, the peptide produced by processing has a length which is suitable for binding to an HLA molecule, in particular a class I HLA molecule, and preferably is 7-20 amino acids in length, more preferably 7-12 amino acids in length, more preferably 8-11 amino acids in length, in particular 9 or 10 amino acids in length. Preferably, the sequence of the peptide which is to be presented following processing is derived from the amino acid sequence of an antigen, i.e., its sequence substantially corresponds and is preferably completely identical to a fragment of an antigen.

Peptides having amino acid sequences substantially corresponding to a sequence of a peptide which is presented by the class I HLA may differ at one or more residues that are not essential for T cell receptor (TCR) recognition of the peptide as presented by the class I HLA, or for peptide binding to HLA. Such substantially corresponding peptides are also capable of stimulating an antigen-responsive CTL and may be considered immunologically equivalent.

An antigen peptide when presented by HLA should be recognizable by a T cell receptor. Preferably, the antigen peptide if recognized by a T cell receptor is able to induce in the presence of appropriate co-stimulatory signals, clonal expansion of the T cell carrying the T cell receptor specifically recognizing the antigen peptide. Preferably, antigen peptides, in particular if presented in the context of HLA molecules, are capable of stimulating an immune response, preferably a cellular response against the antigen from which they are derived or cells characterized by expression of the antigen and preferably characterized by presentation of the antigen. Preferably, an antigen peptide is capable of stimulating a cellular response against a cell characterized by presentation of the antigen with class I HLA and preferably is capable of stimulating an antigen-responsive CTL. Activation and/or expansion of CD4+ or CD8+ T cells may be determined by any method known in the art, including any method described herein.

The term “epitope” refers to an antigenic determinant in a molecule such as an antigen, i.e., to a part in or fragment of the molecule that is recognized by the immune system, for example, that is recognized by a T cell, in particular when presented in the context of HLA molecules. An epitope of a protein such as a tumour antigen preferably comprises a continuous or discontinuous portion of said protein and is preferably between 5 and 100, preferably between 5 and 50, more preferably between 8 and 30, most preferably between 10 and 25 amino acids in length, for example, the epitope may be preferably 9, 10, 11, 12, 13, 14, 15, 16, 17, 18, 19, 20, 21, 22, 23, 24, or 25 amino acids in length. It is particularly preferred that the epitope in the context of the present invention is a T cell epitope.

Cancer/Tumour cells

The modified tumour cells used in the present invention are prepared from tumour cells, e.g., obtained from tumours, or tissue or body fluids containing tumour cells, surgically resected or retrieved in the course of a treatment for a cancer. The ethanol-treated tumour cells are useful in the preparation of, e.g., tumour cell vaccines for treating cancer, including metastatic and primary cancers. If used in a tumour cell vaccine, the preserved tumour cells should be incapable of growing and dividing after administration into the subject, such that they are dead or substantially in a state of no growth. It is to be understood that “dead cells” means a cell which do not have an intact cell or plasma membrane and that will not divide in vivo; and that “cells in a state of no growth” means live cells that will not divide in vivo. Conventional methods of suspending cells in a state of no growth are known to skilled artisans and may be useful in the present invention. For example, cells may be irradiated prior to use such that they do not multiply. Tumour cells may be irradiated to receive a dose of 2500 cGy to prevent the cells from multiplying after administration. Alternatively, ethanol treatment may result in dead cells.

The tumour cells can be prepared from virtually any type of tumour. The present invention contemplates the use of tumour cells from solid tumours, including carcinomas; and non-solid tumours, including hematologic malignancies. Examples of solid tumours from which tumour cells can be derived include sarcomas and carcinomas such as, but not limited to: fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumour, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumour, cervical cancer, testicular tumour, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma. Hematologic malignancies include leukemias, lymphomas, and multiple myelomas. The following are non-limiting preferred examples of tumour cells to be preserved according to the present invention: melanoma, including stage-4 melanoma; ovarian, including advanced ovarian; small cell lung cancer; leukemia, including and not limited to acute myelogenous leukemia; colon, including colon metastasized to liver; rectal, colorectal, breast, lung, kidney, and prostate cancer cells.

Tumour cell vaccines can be prepared from any of the tumour cell types listed above. Such tumour cell vaccines can comprise preserved cells, i.e., cells treated with ethanol according to the method of the invention. Preferably, the vaccine comprises the same type of cells as the tumour to be treated. Most preferably, the tumour cells are autologous, derived from the patient for whom treatment with the vaccine is intended. Vaccines comprising tumour cells prepared using the method of the invention can used for treatment of both solid and non-solid tumours, as exemplified above. Thus, the invention includes “preserved” vaccines prepared from, and intended for treatment of, solid tumours, including carcinomas; and non-solid tumours, including hematologic malignancies. Preferred tumour types for vaccines include melanoma, ovarian cancer, colon cancer, and small cell lung cancer.

The tumour cells are preferably of the same type as, most preferably syngeneic (e.g., autologous or tissue-type matched) to, the cancer which is to be treated. For purposes of the present invention, syngeneic refers to tumour cells that are closely enough related genetically that the immune system of the intended recipient will recognize the cells as “self”, e.g., the cells express the same or almost the same complement of HLA molecules. Another term for this is “tissue-type matched.” For example, genetic identity may be determined with respect to antigens or immunological reactions, and any other methods known in the art. Preferably the cells originate from the type of cancer which is to be treated, and more preferably, from the same patient who is to be treated. The tumour cells can be, although not limited to, autologous cells dissociated from biopsy or surgical resection specimens, or from tissue culture of such cells. Nonetheless, allogeneic cells and stem cells are also within the scope of the present invention.

Tumour cells for use in the present invention may be prepared as follows. Tumours are processed as described by Berd et al. (Cancer Res. 1986;46:2572; see also U.S. Pat. No. 5,290,551; U.S. patent application Ser. No. 08/203,004, U.S. patent application Ser. No. 08/475,016, and U.S. patent application Ser. No. 08/899,905). The cells are extracted by dissociation, such as by enzymatic dissociation with collagenase, or, alternatively, DNase, or by mechanical dissociation such as with a blender, teasing with tweezers, mortar and pestle, cutting into small pieces using a scalpel blade, and the like. Mechanically dissociated cells can be further treated with enzymes as set forth above to prepare a single cell suspension.

Tumour cells may also be prepared according to Hanna et al., U.S. Pat. No. 5,484,596. Briefly, tumour tissue is obtained from patients suffering from the particular solid cancer from which the vaccine is to be prepared. The tumour tissue is surgically removed from the patient, separated from any non-tumour tissue, and cut into small pieces, e.g., fragments 2-3 mm in diameter. The tumour fragments are then digested to free individual tumour cells by incubation in an enzyme solution. After digestion, the cells are pooled and counted, and cell viability is assessed. If desired, a Trypan Blue exclusion test can be used to assess cell viability.

In addition, tumour cells can be prepared according to the following procedure (see Hanna et al., U.S. Pat. No. 5,484,596). The tissue dissociation procedure of Peters et al. (Cancer Research 1979;39:1353-1360) can be employed using sterile techniques throughout under a laminar flow hood. Tumour tissue can be rinsed three times in the centrifuge tube with HBSS and gentamicin and transferred to a petri dish on ice. Scalpel dissection removed extraneous tissue and the tumour are minced into pieces approximately 2 to 3 mm in diameter. Tissue fragments are placed in a 75 ml flask with 20-40 ml of 0.14% (200 units/mil) Collagenase Type 1 (Sigma C-0130) and 0.1% (500 Kunitz units/ml) deoxyribonuclease type 1 (Sigma D-0876) (DNAase 1, Sigma D-0876) prewarmed to 37° C. Flasks are placed in a 37° C. water bath with submersible magnetic stirrers at a speed which cause tumbling, but not foaming. After a 30-minute incubation, free cells are decanted through three layers of sterile medium-wet nylon mesh (166t: Martin Supply Co., Baltimore, Md.) into a 50 ml centrifuge tube. The cells are centrifuged at 1200 rpm 250×g) in a refrigerated centrifuge for 10 minutes. The supernatant is poured off and the cells are resuspended in 5-10 ml of DNase (0.1% in HBSS) and held at 37° C. for 5-10 minutes. The tube is filled with HBSS, washed by centrifugation, resuspended to 15 ml in HBSS and held on ice. The procedure is repeated until sufficient cells are obtained, usually three times for tumour cells. Cells from the different digests are then pooled, counted. Optionally, although not necessarily, cell viability is assessed by the Trypan Blue exclusion test.

Cancer cells or cell lines obtained as described may be combined directly with the other components of the vaccine. However, it is preferable to inactivate the cancer cells to prevent further proliferation once administered to the subject. Any physical, chemical, or biological means of inactivation may be used, including but not limited to irradiation (preferably with at least about 5,000 cGy, more preferably at least about 10,000 cGy, even more preferably at least about 20,000 cGy); or treatment with mitomycin-C (preferably at least 10 μg/mL; more preferably at least about 50 μg/mL).

Cancer cells for use as a tumour antigen source may alternatively be fixed with such agents as glutaraldehyde, paraformaldehyde, or formalin. They may also be in an ionic or non-ionic detergent, such as deoxycholate or octyl glucoside, or treated, for example, using Vaccinia Virus or Newcastle Disease Virus. If desired, solubilized cell suspensions may be clarified or subjected to any of a number of standard biochemical separation procedures to enrich or isolate particular tumour-associated antigens or plurality of antigens. Preferably, tumour antigen associated with the outer membrane of tumour cells, or a plurality of tumour associated antigens is enriched. The degree of enrichment may be 10-fold or more preferably 100-fold over that of a whole-cell lysate. Isolated antigens, recombinant antigens, or mixtures thereof may also be used. Before combination with other components of the vaccine, the tumour antigen preparation is depleted of the agent used to treat it; for example, by centrifuging and washing the fixed cells, or dialysis of the solubilized suspension. Preparation of tumour antigen, particularly beyond inactivation of the source tumour cell, may be viewed as optional and unnecessary for the practice of the embodiments of the invention, unless specifically required.

Autologous Cells

The use of autologous cytokine-expressing cells in a vaccine of the invention provides advantages since each patient's tumour expresses a unique set of tumour antigens that can differ from those found on histologically-similar, MHC-matched tumour cells from another patient. See, e.g., Kawakami et al., J. Immunol., 148, 638-643 (1992); Darrow et al., J. Immunol., 142, 3329-3335 (1989); and Hom et al., J. Immunother., 10, 153-164 (1991). In contrast, MHC-matched tumour cells provide the advantage that the patient need not be taken to surgery to obtain a sample of their tumour for vaccine production.

In one preferred aspect, the present invention comprises a method of treating cancer by carrying out the steps of: (a) obtaining tumour cells from a mammal, preferably a human, harboring a tumour; (b) modifying the tumour cells to render them capable of producing a cytokine or an increased level of a cytokine naturally produced by the cells and at least one additional cancer therapeutic agent relative to unmodified tumour cells; (c) rendering the modified tumour cells proliferation incompetent; and (d) readministering the modified tumuor cells to the mammal from which the tumour cells were obtained or to a mammal with the same MHC type as the mammal from which the tumour cells were obtained. The administered tumour cells are autologous or MHC-matched to the host.

The same autologous tumour cells may express both a cytokine and cancer therapeutic agent(s) or a cytokine and one or more cancer therapeutic agent(s) may be expressed by a different autologous tumour cell population. In one aspect of the invention, an autologous tumor cell is modified by introduction of a vector comprising a nucleic acid sequence encoding a cytokine, operably linked to a promoter and expression/control sequences necessary for expression thereof. In another aspect, the same autologous tumour cell is modified by introduction of a vector comprising a nucleic acid sequence encoding at least one additional cancer therapeutic agent operably linked to a promoter and expression/control sequences necessary for expression thereof. In a further aspect, a second autologous tumour cell is modified by introduction of a vector comprising a nucleic acid sequence encoding at least one additional cancer therapeutic agent operably linked to a promoter and expression/control sequences necessary for expression thereof. The nucleic acid sequence encoding the cytokine and additional cancer therapeutic agent(s) may be introduced into the same or a different autologous tumour cell using the same or a different vector. The nucleic acid sequence encoding the cytokine or cancer therapeutic agent may or may not further comprise a selectable marker sequence operably linked to a promoter.

Allogeneic Cells

Researchers have sought alternatives to autologous and MHC-matched cells as tumour vaccines, as reviewed by Jaffee et al., Seminars in Oncology, 22, 81-91 (1995). Early tumour vaccine strategies were based on the understanding that the vaccinating tumour cells function as the antigen presenting cells (APCs) and present tumour antigens by way of their MHC class I and II molecules, and directly activate the T cell arm of the immune system. The results of Huang et al. (Science, 264, 961-965, 1994), indicate that professional APCs of the host rather than the vaccinating tumour cells prime the T cell arm of the immune system by secreting cytokine(s) such as GM-CSF such that bone marrow-derived APCs are recruited to the region of the tumour. The bone marrow-derived APCs take up the whole-cellular protein of the tumour for processing, and then present the antigenic peptide(s) on their MHC class I and II molecules, thereby priming both the CD4+ and the CD8+ T cell arms of the immune system, resulting in a systemic tumour-specific anti-tumour immune response. These results suggest that it may not be necessary or optimal to use autologous or MHC-matched tumour cells in order to elicit an anti-cancer immune response and that the transfer of allogeneic MHC genes (from a genetically dissimilar individual of the same species) can enhance tumour immunogenicity. More specifically, in certain cases, the rejection of tumours expressing allogeneic MHC class I molecules resulted in enhanced systemic immune responses against subsequent challenge with the unmodified parental tumour, as reviewed in Jaffee et al., supra, and Huang et al., supra.

As described herein, a “tumour cell line” comprises cells that were initially derived from a tumour. Such cells typically are transformed (i.e., exhibit indefinite growth in culture).

In one preferred aspect, the invention provides a method for treating cancer by carrying out the steps of: (a) obtaining a tumour cell line; (b) modifying the tumour cell line to render the cells capable of producing an increased level of a cytokine alone or in combination with at least one additional cancer therapeutic agent relative to the unmodified tumour cell line; (c) rendering the modified tumour cell line proliferation incompetent; and (d) administering the tumour cell line to a mammalian host having at least one tumour that is the same type of tumour as that from which the tumour cell line was obtained or wherein the tumour cell line and host tumour express at least one common antigen. The administered tumour cell line is allogeneic and is not MHC-matched to the host. Such allogeneic lines provide the advantage that they can be prepared in advance, characterized, aliquoted in vials containing known numbers of cytokine-expressing cells and stored such that well characterize cells are available for administration to the patient. Methods for the production of gene-modified allogeneic cells are described for example in WO 00/72686A1, expressly incorporated by reference herein.

In one approach to preparing a cytokine-expressing cellular vaccine comprising gene-modified allogeneic cells, cytokine and cancer therapeutic agent-encoding nucleic acid sequences are introduced into a cell line that is an allogeneic tumour cell line (i.e., derived from an individual other than the individual being treated). In another approach, cytokine and cancer therapeutic agent encoding nucleic acid sequences are introduced into separate (i.e. different) allogeneic tumour cell lines. The cell or population of cells may be from a tumour cell line of the same type as the tumour or cancer being treated. The tumour and/or tumour cell line may be from any form of cancer, including, but not limited to, carcinoma of the bladder, breast, colon, kidney, liver, lung, ovary, cervix, pancreas, rectum, prostate, stomach, epidermis; a hematopoietic tumour of lymphoid or myeloid lineage; a tumour of mesenchymal origin such as a fibrosarcoma or rhabdomyosarcoma; or another tumour, including a melanoma, teratocarcinoma, neuroblastoma, glioma, adenocarcinoma and non-small lung cell carcinoma.

In one aspect of the invention, the allogeneic tumour cell is modified by introduction of a vector comprising a nucleic acid sequence encoding a cytokine, operably linked to a promoter and expression control sequences necessary for expression thereof. In another aspect, the same allogeneic tumour cell or a second allogeneic tumour cell is modified by introduction of a vector comprising a nucleic acid sequence encoding at least one additional cancer therapeutic agent operably linked to a promoter and expression control sequences necessary for expression thereof. The nucleic acid sequence encoding the cytokine and additional cancer therapeutic agent(s) may be introduced into the same or a different allogeneic tumour cell using the same or a different vector. The nucleic acid sequence encoding the cytokine or cancer therapeutic agent may or may not further comprise a selectable marker sequence operably linked to a promoter.

Genetic alteration of a cell may be effected by any method known in the art. Typically, an encoding sequence for the desired cytokine is operatively linked to a heterologous promoter that will be constitutively or inducibly active in the target cell, along with other controlling elements and a poly-A sequence necessary for transcription and translation of the protein. The expression cassette thus composed is introduced into the cell by any method known in the art, such as calcium-phosphate precipitation, insertion using cationic liposomes, or using a viral vector tropic for the cells. Methods of genetic alteration are described in the patent publications cited in relation to some of the cytokines listed earlier.

One preferred method is the use of adenovirus vectors, a method with which the skilled person will be familiar. Briefly, adenovial recombinant expression vectors prepared by genetic engineering of commercially available plasmids. Suitable infection conditions and multiplicities of infection (MOI) may be determined in preliminary experiments using a reporter gene such as β-galactosidase, and then used for cytokine transfer. An advantage of using a viral vector is that the vector may first be replicated, and then an entire population of cells may be infected and altered. Accordingly, genetically altered cytokine secreting cells may be established as a cell line, or a freshly obtained cell isolate or cell culture is altered de novo just prior to use in a vaccine of this invention In the latter instance, preparation of the vaccine would additionally comprise the step of transducing a population of cells allogeneic to the intended recipient with a vector comprising an encoding region for a particular cytokine of interest. Transduction using adenoviral vectors and the like is especially preferred when it is desirable to achieve very high levels of cytokine expression by the genetically altered cells.

Antigen Presenting Cells

“Antigen presenting cells” (APC) are cells which present peptide fragments of protein antigens in association with HLA molecules on their cell surface. Some APCs may activate antigen specific T cells. Antigen presentation typically stimulates T cells to become either “cytotoxic” CD8+ cells or “helper” CD4+ cells.

As used herein, the term “professional antigen presenting cell” refers to APCs which specialize in presenting antigen to T cells. Examples of professional APCs include dendritic cells, macrophages and B cells. Professional APCs are very efficient at internalizing antigens, either by phagocytosis or by receptor-mediated endocytosis, processing the antigen into peptide fragments and then displaying those peptides, bound to a class II MHC molecule, on their membrane. The expression of co-stimulatory molecules and MHC class II are defining features of professional APCs. All professional APCs also express MHC class I molecules as well.

“Antigen processing” or “processing” refers to the degradation of a polypeptide or antigen into procession products, which are fragments of said polypeptide or antigen (e.g., the degradation of a polypeptide into peptides) and the association of one or more of these fragments (e.g., via binding) with HLA molecules for presentation by cells, preferably antigen presenting cells, to specific T cells.

Although many cell types can function as an antigen-presenting cell, certain cells are “professional antigen presenting cells”. Professional antigen-presenting cells, including macrophages, B cells and dendritic cells, present foreign antigens to helper T cells, while other cell types can present antigens originating inside the cell to cytotoxic T cells. In addition to the MHC family of proteins, antigen presentation relies on other specialized signaling molecules on the surfaces of both APCs and T cells. The main types of professional antigen-presenting cells are dendritic cells, which have the broadest range of antigen presentation, and are probably the most important antigen-presenting cells, macrophages, B-cells, and certain activated epithelial cells.

Dendritic cells (DCs) are leukocyte populations that present antigens captured in peripheral tissues to T cells via both HLA class II and I antigen presentation pathways. It is well known that dendritic cells are potent inducers of immune responses and the activation of these cells is a critical step for the induction of antitumoural immunity.

T Cells

Anatomic sources of leukocytes, preferably T cells, from a subject include peripheral blood, tumours, malignant effusions, and draining lymph nodes. Lymphocytes used for adoptive transfer can either be derived from the stroma of resected tumours (tumour infiltrating lymphocytes), or from blood and: genetically engineered to express antitumour T cell receptors or chimeric antigen receptors (CARs), enriched with mixed lymphocyte tumour cell cultures (MLTCs) or cloned using autologous antigen presenting cells and tumour derived peptides. The lymphocytes used for infusion can be isolated from an allogenic donor, preferably HLA matched, or from the cancer-bearing subject. In one embodiment, the leukocytes, preferably T cells, from a subject are not obtained or derived from the bone marrow.

In any method of the invention the leukocytes, preferably T cells that have been cultured in the presence of a tumour antigen can be transferred into the same mammal from which cells were obtained. In other words, the cells used in a method of the invention can be an autologous cell, i.e., can be obtained from the mammal in which the medical condition is treated or prevented. Alternatively, the cell can be allogenically transferred into another subject. Preferably, the cell is autologous to the subject in a method of treating or preventing a medical condition in the subject.

T cells may be obtained using routine cell sorting techniques that discriminate and segregate T cells based on T cell surface markers can be used to obtain an isolated population, for example of CD8+ T cells for use in the compositions and methods of the invention. For example, a biological sample including blood and/or peripheral blood lymphocytes can be obtained from an individual and CD8+ T cells isolated from the sample using commercially available devices and reagents, thereby obtaining an isolated population of CD8+ T cells. Murine CD8+ T cells may be further characterized and/or isolated on a phenotypic basis via the use of additional cell surface markers such as CD44, L-selectin (CD62L), CD25, CD49d, CD122, CD27, CD43, CD69, KLRG-1, CXCR3, CCR7, IL-7Ra and KLRG-1. CD8+ T cells may be initially enriched by negatively selecting CD4+, NK1.1+, B220+, CD11b+, TER119+, Gr-1+, CD11c+ and CD19+ cells. Naive CD8+ T cells are characterized as CD44 low, CD62L high, CCR7 high, CD25 low, CD43 low, CD49d low, CD69 low, IL-7Ra high and CD122 low, whereas antigen experienced memory T cells are CD44 high, CD49d high, CD122 high, CD27 high, CD43 high and CXCR3 high. Memory CD8+ CD44 high T cells can be further sub-divided into lymphoid-tissue residing Central Memory T cells (CD62L high, CCR7 high) and non-lymphoid tissue residing Effector Memory T cells (CD62L low, CCR7 low) (Klonowski et al. Immunity 2004, 20:551-562). The isolated population of CD8+ T cells can be mixed with the antigen in any suitable container, device, cell culture media, system, etc., and can be cultured in vitro and/or exposed to the one or more antigens, and any other reagent, or cell culture media, in order to expand and/or mature and/or differentiate the T cells to have any of various desired cytotoxic T cell characteristics.

Human CD8+ T-cell types and/or populations can be identified using the phenotypic cell-surface markers CD62L, CCR7, CD27, CD28 and CD45RA or CD45RO (Sallusto F et al. Nature 1999, 401:708-712). As used herein, CD8+ T-cell types and/or populations have the following characteristics or pattern of expression of cell surface markers: Naive T cells are characterized as CD45RA+, CD27+, CD28+, CD62L+ and CCR7+; CD45RO+ Central Memory T cells are CD45RA−, CD27+, CD28+, CD62L+ and CCR7+; CD45RO+ Effector Memory T cells are defined by the lack of expression of these five markers (CD45RA−, CD27−, CD28−, CD62L− and CCR7−); and terminally differentiated Effector Memory CD45RA+ T cells are characterized as CD45RP+, CCR7−, CD27−, CD28−, CD62L−. Terminally differentiated Effector Memory cells further up-regulate markers such as CD57, KLRG1, CX3CR1 and exhibit strong cytotoxic properties characterized by their ability to produce high levels of Granzyme A and B, Perforin and IFNγ. Therefore, various populations of T cells can be separated from other cells and/or from each other based on their expression or lack of expression of these markers. In this manner, the invention provides methods of separating different populations of CD8+ T cells and also separated or isolated populations of CD8+ T cells. The CD8+ T cell types described herein may also be isolated by any other suitable method known in the art; for example, if a particular antigen or antigens are used to produce antigen-specific CD8+ T cells, those cells can be separated or isolated from other cells by affinity purification using that antigen or antigens; appropriate protocols are known in the art.

Different CD8+ T cell types can also exhibit particular functions, including, for example: secretion of IFN-γ; secretion of IL-2; production of Granzyme B; expression of FasL and expression of CD 107. However, while the expression pattern of cell surface markers is considered diagnostic of each particular CD8+ T cell type and/or population as described herein, the functional attributes of each cell type and/or population may vary depending on the amount of stimulation the cell(s) has or have received.

Effector functions or properties of T cells can be determined by the effector molecules that they release in response to specific binding of their T-cell receptor with antigen:MHC complex on the target cell, or in the case of CAR T-cells interaction of the chimeric antigen receptor, e.g. scFv, with the antigen expressed on the target cell. Cytotoxic effector molecules that can be released by cytotoxic CD8+ T cells include perforin, granzymes A and B, granulysin and Fas ligand. Generally, upon degranulation, perforin inserts itself into the target cell's plasma membrane, forming a pore, granzymes are serine proteases which can trigger apoptosis (a form of cell death), granulysin induces apoptosis in target cells, and Fas ligand can also induce apoptosis. Typically, these cytotoxic effector molecules are stored in lytic granules in the cell prior to release. Other effector molecules that can be released by cytotoxic T cells include IFN-γ, TNF-β and TNF-α. IFN-γ can inhibit viral replication and activate macrophages, while TNF-β and TNF-α can participate in macrophage activation and in killing target cells. In any method of the invention, before administration or reintroduction of the cells contacted with a tumour antigen, those cells will be assessed for their cytotoxic activity by flow cytometry using fluorochrome-conjugated antibodies against surface and intracellular markers that specify cytotoxic effector T cells including Granzyme A and B, Perforin and IFNγ.

An activated T cell is a cell that is no longer in GO phase, and begins to produce one or more cytotoxins, cytokines and/or other membrane-associated markers characteristic of the cell type (e.g., CD8+) as described herein and is capable of recognizing and binding any target cell that displays the particular peptide:MHC complex or antigen alone on its surface and releasing its effector molecules.

Natural Killer Cells

Natural killer (NK) cells are innate lymphocytes important for mediating anti-viral and anti-cancer immunity through cytokine and chemokine secretion, and through the release of cytotoxic granules.

Antibody-dependent cellular cytotoxicity and antibody-dependent cytokine/chemokine production are primarily mediated by the specialized subset of lymphocytes, natural killer (NK) cells. NK cells are effector cells that comprise the third largest population of lymphocytes and are important for host immuno-surveillance against tumor and pathogen-infected cells.

Upon activation, NK cells produce cytokines and chemokines abundantly and at the same time exhibit potent cytolytic activity. Activation of NK cells can occur through the direct binding of NK cell receptors to ligands on the target cell, as seen with direct tumor cell killing, or through the crosslinking of the Fc receptor (CD16; FcyRIII) by binding to the Fc portion of antibodies bound to an antigen-bearing cell. This CD16 engagement (CD16 crosslinking) initiates NK cell responses via intracellular signals that are generated through one, or both, of the CD16-associated adaptor chains, FcRy or CD3ζ. Triggering of CD16 leads to phosphorylation of the γ or ζ chain, which in turn recruits tyrosine kinases, syk and ZAP-70, initiating a cascade of signal transduction leading to rapid and potent effector functions. The most well-known effector function is the release of cytoplasmic granules carrying toxic proteins to kill nearby target cells through the process of antibody-dependent cellular cytotoxicity. CD16 crosslinking also results in the production of cytokines and chemokines that, in turn, activate and orchestrate a series of immune responses.

This release of cytokines and chemokines can play a role in the anti-cancer activity of NK cells in vivo. NK cells also have small granules in their cytoplasm containing perforin and proteases (granzymes). Upon release from the NK cell, perforin forms pores in the cell membrane of targeted cells through which the granzymes and associated molecules can enter, inducing apoptosis. The fact that NK cells induce apoptosis rather than necrosis of target cells is significant—necrosis of a virus-infected cell would release the virions, whereas apoptosis leads to destruction of the virus inside the cells.

Natural killer cells can be identified by any convenient procedure, for example, by their expression patterns. For example, mature NK cells express known markers that can be detected by procedures available in the art. A typical human marker profile includes, for example, NKG2A, NKG2D, NKp30, NKp44, NKp46, CD56, CD161, 2B4, NTB-A, CRACC, DNAM-1, CD69, CD25 and/or NKp44. Other markers for natural killer cells include KIRs. A typical mouse marker profile includes, for example, NK1.1, CD122, LY49 Family (Ly49A, Ly49C, Ly49D, Ly49E, Ly49F, Ly49G, Ly49H, and Ly49I), and/or NKG2A/C/E. NK cells do not express T-cell antigen receptors (TCR), CD3 or surface immunoglobulins (Ig) B cell receptor, but generally express the surface marker CD56 in humans.

Conditions to be Treated

The specification describes any metastatic or non-metastatic tumour, cancer, malignancy or neoplasia of any cell or tissue origin. The tumour may be in any stage, e.g., a stage I, II, Ill, IV or V tumour, or in remission.

As used herein, the terms “tumour,” “cancer,” “malignancy,” and “neoplasia” are used interchangeably and refer to a cell or population of cells whose growth, proliferation or survival is greater than growth, proliferation or survival of a normal counterpart cell, e.g. a cell proliferative or differentiative disorder. Such disorders can affect virtually any cell or tissue type, e.g., carcinoma, sarcoma, melanoma, neural, and reticuloendothelial or haematopoietic neoplastic disorders (e.g., myeloma, lymphoma or leukemia). A tumour can arise from a multitude of primary tumour types, including but not limited to breast, lung, thyroid, head and neck, brain, lymphoid, gastrointestinal (mouth, esophagus, stomach, small intestine, colon, rectum), genitourinary tract (uterus, ovary, cervix, bladder, testicle, penis, prostate), kidney, pancreas, liver, bone, muscle, skin, and metastasize to other secondary sites.

Cells comprising a tumour may be aggregated in a cell mass or be dispersed. A “solid tumour” refers to neoplasia or metastasis that typically aggregates together and forms a mass. Specific examples include visceral tumours such as melanomas, breast, pancreatic, uterine and ovarian cancers, testicular cancer, including seminomas, gastric or colon cancer, hepatomas, adrenal, renal and bladder carcinomas, lung, head and neck cancers and brain tumours/cancers.

Carcinomas refer to malignancies of epithelial or endocrine tissue, and include respiratory system carcinomas, gastrointestinal system carcinomas, genitourinary system carcinomas, testicular carcinomas, breast carcinomas, prostatic carcinomas, endocrine system carcinomas, and melanomas. Melanoma refers to malignant tumours of melanocytes and other cells derived from pigment cell origin that may arise in the skin, the eye (including retina), or other regions of the body, including the cells derived from the neural crest that also gives rise to the melanocyte lineage. A pre-malignant form of melanoma, known as dysplastic nevus or dysplastic nevus syndrome, is associated with melanoma development.

Exemplary carcinomas include those forming from the uterine cervix, lung, prostate, breast, head and neck, colon, pancreas, testes, adrenal, kidney, esophagus, stomach, liver and ovary. The term also includes carcinosarcomas, e.g., which include malignant tumours composed of carcinomatous and sarcomatous tissues. Adenocarcinoma includes a carcinoma of a glandular tissue, or in which the tumour forms a gland like structure.

Sarcomas refer to malignant tumours of mesenchymal cell origin. Exemplary sarcomas include for example, lymphosarcoma, liposarcoma, osteosarcoma, chondrosarcoma, leiomyosarcoma, rhabdomyosarcoma and fibrosarcoma.

Neural neoplasias include glioma, glioblastoma, meningioma, neuroblastoma, retinoblastoma, astrocytoma, oligodendrocytoma.

A “liquid tumour” refers to neoplasia of the reticuloendothelial or haematopoetic system, such as a lymphoma, myeloma and leukemia, or neoplasia that is diffuse in nature, as they do not typically form a solid mass. Particular examples of leukemias include acute and chronic lymphoblastic, myeolblastic and multiple myeloma. Typically, such diseases arise from poorly differentiated acute leukemias, e.g., erythroblastic leukemia and acute megakaryoblastic leukemia. Specific myeloid disorders include, but are not limited to, acute promyeloid leukemia (APML), acute myelogenous leukemia (AML) and chronic myelogenous leukemia (CML); lymphoid malignancies include, but are not limited to, acute lymphoblastic leukemia (ALL), which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Specific malignant lymphomas include, non-Hodgkin lymphoma and variants, peripheral T cell lymphomas, adult T cell leukemia/lymphoma (ATL), cutaneous T-cell lymphoma (CTCL), large granular lymphocytic leukemia (LGF), Hodgkin's disease and Reed-Stemberg disease.

The immunogenic composition or modified cancer cell of the present invention as described herein are useful in the treatment or prevention of any hyperproliferative condition. An example of a condition is cancer. Exemplary cancers include cystic and solid tumours, bone and soft tissue tumours, including tumours in anal tissue, bile duct, bladder, blood cells, bowel, brain, breast, carcinoid, cervix, eye, esophagus, head and neck, kidney, larynx, leukemia, liver, lung, lymph nodes, lymphoma, melanoma, mesothelioma, myeloma, ovary, pancreas, penis, prostate, skin (e.g. squamous cell carcinoma), sarcomas, stomach, testes, thyroid, vagina, vulva. Soft tissue tumours include Benign schwannoma Monosomy, Desmoid tumour, lipo-blastoma, lipoma, uterine leiomyoma, clear cell sarcoma, dermatofibrosarcoma, Ewing sarcoma, extraskeletal myxoid chondrosarcoma, liposarcooma myxoid, Alveolar rhabdomyosarcoma and synovial sarcoma. Specific bone tumours include nonossifying fibroma, unicameral bone cyst, enchon-droma, aneurismal bone cyst, osteoblastoma, chondroblastoma, chondromyxofibroma, ossifying fibroma and adamantinoma, Giant cell tumour, fibrous dysplasia, Ewing's sarcoma eosinophilic granuloma, osteosarcoma, chondroma, chondrosarcoma, malignant fibrous histiocytoma and metastatic carcinoma. Leukemias include acute lymphoblastic, acute myeloblastic, chronic lymphocytic and chronic myeloid.

Other examples include breast tumours, colorectal tumours, adenocarcinomas, mesothelioma, bladder tumours, prostate tumours, germ cell tumour, hepatoma/cholongio, carcinoma, neuroendocrine tumours, pituitary neoplasm, small round cell tumour, squamous cell cancer, melanoma, atypical fibroxanthoma, seminomas, nonseminomas, stromal leydig cell tumours, Sertoli cell tumours, skin tumours, kidney tumours, testicular tumours, brain tumours, ovarian tumours, stomach tumours, oral tumours, bladder tumours, bone tumours, cervical tumours, esophageal tumours, laryngeal tumours, liver tumours, lung tumours, vaginal tumours and Wilm's tumour.

The words ‘treat’ or ‘treatment’ refer to therapeutic treatment wherein the object is to slow down (lessen) an undesired physiological change or disorder. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. Treatment' can also mean prolonging survival as compared to expected survival if not receiving treatment. Treatment may not necessarily result in the complete clearance of cancer or infected cells but may reduce or minimise complications and side effects of infection, or the presence or progression of cancer. The success or otherwise of treatment may be monitored by physical examination of the subject, cytopathological, serological DNA, or mRNA detection techniques.

“Preventing”, “prevention”, “preventative” or “prophylactic” refers to keeping from occurring, or to hinder, defend from, or protect from the occurrence of a condition, disease, disorder, or phenotype, including an abnormality or symptom. A subject in need of prevention may be prone to develop the condition.

The present invention also includes methods of preventing the development of cancer in a subject. For example the subject for whom prevention of cancer is required may be considered to be at risk of developing cancer, but does not yet have detectable cancer. A subject at risk of the development of cancer may be a subject with a family history of cancer, and/or a subject for whom genetic testing or other testing indicates a high risk or high likelihood of the development of cancer. The subject may have cancer stem cells but does not yet have any detectable tumours. It will be understood that methods of preventing the development of cancer include methods of delaying the onset of cancer in a subject.

The term “ameliorate” or “amelioration” refers to a decrease, reduction or elimination of a condition, disease, disorder, or phenotype, including an abnormality or symptom. A subject in need of treatment may already have the condition, or may be prone to have the condition or may be one in whom the condition is to be prevented.

In some examples, a method of the present invention comprises administering a prophylactically or therapeutically effective amount of a composition of the invention as described herein.

The phrase ‘therapeutically effective amount’ generally refers to an amount of a compound of the present invention that (i) treats the particular disease, condition, or disorder, (ii) attenuates, ameliorates, or eliminates one or more symptoms of the particular disease, condition, or disorder, or (iii) delays the onset of one or more symptoms of the particular disease, condition, or disorder described herein. The exact dose will depend on the purpose of the treatment, and will be ascertainable by one skilled in the art using known techniques. As is known in the art and described above, adjustments for systemic versus localized delivery, age, body weight, general health, sex, diet, time of administration, drug interaction and the severity of the condition may be necessary, and will be ascertainable with routine experimentation by those skilled in the art.

As used herein, the term “prophylactically effective amount” shall be taken to mean a sufficient quantity of a protein to prevent or inhibit or delay the onset of one or more detectable symptoms of a clinical condition. The skilled artisan will be aware that such an amount will vary depending on, for example, the specific antigen binding site(s) administered and/or the particular subject and/or the type or severity or level of condition and/or predisposition (genetic or otherwise) to the condition. Accordingly, this term is not to be construed to limit the present invention to a specific quantity, e.g., weight or amount of antigen binding site(s), rather the present invention encompasses any amount of the antigen binding site(s) sufficient to achieve the stated result in a subject.

Suitable dosages of a composition of the invention as described herein will vary depending on the specific the condition to be treated and/or the subject being treated. It is within the ability of a skilled physician to determine a suitable dosage, e.g., by commencing with a sub-optimal dosage and incrementally modifying the dosage to determine an optimal or useful dosage. Alternatively, to determine an appropriate dosage for treatment/prophylaxis, data from the cell culture assays or animal studies are used, wherein a suitable dose is within a range of circulating concentrations that include the ED50 of the active compound with little or no toxicity. The dosage may vary within this range depending upon the dosage form employed and the route of administration utilized. A therapeutically/prophylactically effective dose can be estimated initially from cell culture assays. A dose may be formulated in animal models to achieve a circulating plasma concentration range that includes the IC₅₀ (i.e., the concentration or amount of the compound which achieves a half-maximal inhibition of symptoms) as determined in cell culture. Such information can be used to more accurately determine useful doses in humans. Levels in plasma maybe measured, for example, by high performance liquid chromatography.

It will be clearly understood that, although this specification refers specifically to applications in humans, the invention is also useful for veterinary purposes. Thus in all aspects the invention is useful for domestic animals such as cattle, sheep, horses and poultry; for companion animals such as cats and dogs; and for zoo animals. Therefore, the general term “subject” or “subject to be/being treated” is understood to include all animals (such as humans, apes, dogs, cats, horses, and cows) that require an enhanced immune response, for example subjects having cancer.

The term “administered” means administration of a therapeutically effective dose of the aforementioned composition of the present invention as described herein to the subject.

Subjects requiring treatment include those already having a benign, pre-cancerous, or non-metastatic tumour as well as those in which the occurrence or recurrence of cancer is to be prevented. Subjects may have metastatic cells, including metastatic cells present in the ascites fluid and/or lymph node.

A subject in need of treatment may be one diagnosed with, or at risk of developing, any one of the cancers described herein.

In any method of the invention, one or more of the following effects may be observed: reduction in the reoccurrence of malignant tumours, reduction in metastasis of malignant tumours, reduction in number or size of tumours, differentiation of tumour cells, expression of β-catenin and E-cadherin in malignant tumours to facilitate cell-to-cell adhesion and reduction in metastasis, reduction in tumour cells ability to prevent immunorecognition.

The objective or outcome of treatment may be to reduce the number of cancer cells; reduce the primary tumour size; inhibit (i.e., slow to some extent and preferably stop) cancer cell infiltration into peripheral organs; inhibit (i.e., slow to some extent and preferably stop) tumour metastasis; inhibit, to some extent, tumour growth; and/or relieve to some extent one or more of the symptoms associated with the disorder.

Efficacy of treatment can be measured by assessing the duration of survival, time to disease progression, the response rates (RR), duration of response, and/or quality of life.

The method is particularly useful for extending time to disease progression.

The method is particularly useful for extending survival of the human, including overall survival as well as progression free survival.

The method is particularly useful for providing a complete response to therapy whereby all signs of cancer in response to treatment have disappeared. This does not always mean the cancer has been cured.

The method is particularly useful for providing a partial response to therapy whereby there has been a decrease in the size of one or more tumours or lesions, or in the extent of cancer in the body, in response to treatment.

The objective or outcome of treatment may be any one or more of the following:

to reduce the number of cancer cells;

reduce the primary tumour size;

inhibit (i.e., slow to some extent and preferably stop) cancer cell infiltration into peripheral organs;

inhibit (i.e., slow to some extent and preferably stop) tumour metastasis; inhibit, to some extent, tumour growth;

relieve to some extent one or more of the symptoms associated with the disorder.

In one embodiment, subjects requiring treatment include those having a benign, pre-cancerous, non-metastatic tumour.

In one embodiment, the cancer is pre-cancerous or pre -neoplastic.

In one embodiment, the cancer is a secondary cancer or metastases. The secondary cancer may be located in any organ or tissue, and particularly those organs or tissues having relatively higher hemodynamic pressures, such as lung, liver, kidney, pancreas, bowel and brain. The secondary cancer may be detected in the ascites fluid and/or lymph nodes.

In one embodiment, the cancer may be substantially undetectable.

“Pre-cancerous” or “pre-neoplasia” generally refers to a condition or a growth that typically precedes or develops into a cancer. A “pre-cancerous” growth may have cells that are characterized by abnormal cell cycle regulation, proliferation, or differentiation, which can be determined by markers of cell cycle.

In one embodiment, the cancer is pre-cancerous or pre-neoplastic.

In one embodiment, the cancer is a secondary cancer or metastases. The secondary cancer may be located in any organ or tissue, and particularly those organs or tissues having relatively higher hemodynamic pressures, such as lung, liver, kidney, pancreas, bowel and brain.

Nucleic Acid Based Expression Systems

The skilled person will appreciate that in order to modify a cell as described herein, to express a tumour antigen and/or a IFN-β agonist (including IFN-β, or a variant, fragment or derivative thereof), it may be necessary to prepare nucleic acid expression constructs for introduction into the cell. The skilled person will be familiar with standard techniques in the art for preparing such nucleic acid constructs and vectors, including the addition or other elements (such as promoters, enhancers, terminators etc) for optimising expression of the tumour antigen and/or IFN-β receptor agonist in the cell.

As used herein, the term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques (see, for example, Goodburn and Maniatis et al., 1988 and Ausubel et al., 1996, both incorporated herein by reference).

The term “expression vector” refers to any type of genetic construct comprising a nucleic acid coding for an RNA capable of being transcribed and then translated into a protein, polypeptide, or peptide. Expression vectors can contain a variety of “control sequences,” which refer to nucleic acid sequences necessary for the transcription and possibly translation of an operably linked coding sequence in a particular host cell. In addition to control sequences that govern transcription and translation, vectors and expression vectors may contain nucleic acid sequences that serve other functions as well and are described infra.

The skilled person will also be able to make use of vectors which include a multiple cloning site (MCS); a nucleic acid region that contains multiple restriction enzyme sites, any of which can be used in conjunction with standard recombinant technology to digest the vector (see, for example, Carbonelli et al., 1999, Levenson et al., 1998, and Cocea, 1997, incorporated herein by reference.) “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

In certain embodiments, a plasmid vector is contemplated for use to transform a cell. In general, plasmid vectors containing replicon and control sequences which are derived from species compatible with the cell are used in connection with these cells. The vector ordinarily carries a replication site, as well as marking sequences which are capable of providing phenotypic selection in transformed cells.

A “promoter” is a control sequence that is a region of a nucleic acid sequence at which initiation and rate of transcription are controlled. It may contain genetic elements at which regulatory proteins and molecules may bind, such as RNA polymerase and other transcription factors, to initiate the specific transcription a nucleic acid sequence. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence.

A promoter generally comprises a sequence that functions to position the start site for RNA synthesis. The skilled person will be familiar with suitable promoters, including those well know such as the TATA box. The skilled person will also be familiar with promoters that lack a TATA box, such as, for example, the promoter for the mammalian terminal deoxynucleotidyl transferase gene and the promoter for the SV40 late genes. Additional promoter elements regulate the frequency of transcriptional initiation.

The spacing between promoter elements frequently is flexible, so that promoter function is preserved when elements are inverted or moved relative to one another. A promoter may or may not be used in conjunction with an “enhancer,” which refers to a cis-acting regulatory sequence involved in the transcriptional activation of a nucleic acid sequence.

A promoter may be one naturally associated with a nucleic acid sequence, as may be obtained by isolating the 5′ non-coding sequences located upstream of the coding segment and/or exon. Such a promoter can be referred to as “endogenous.” Alternatively, the promoter may be heterologous (i.e., not the promoter typically associated with the encoded sequence). It may be desirable to use a heterologous promoter, for example to enable increased levels of expression of the nucleic acid sequence as compared to the “native” level of expression. The skilled person will be familiar with promoters for enhancing expression in vivo.

Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other virus, or prokaryotic or eukaryotic cell, and promoters or enhancers not “naturally occurring,” i.e., containing different elements of different transcriptional regulatory regions, and/or mutations that alter expression. For example, promoters that are commonly used in recombinant DNA construction include the β-lactamase (penicillinase), lactose and tryptophan (trp) promoter systems. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. Nos. 4,683,202 and 5,928,906, each incorporated herein by reference). Furthermore, it is contemplated the control sequences that direct transcription and/or expression of sequences within non-nuclear organelles, such as mitochondria, can be employed as well.

Naturally, it will be important to employ a promoter and/or enhancer that effectively directs the expression of the DNA segment in the organelle, cell type, tissue, organ, or organism chosen for expression. Those of skill in the art of molecular biology generally know the use of promoters, enhancers, and cell type combinations for protein expression, (see, for example Sambrook et al. 2001, incorporated herein by reference). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

Any promoter/enhancer combination (as per, for example, the Eukaryotic Promoter Data Base EPDB, http://www.epd.isb-sib.ch/) could also be used to drive expression. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

The identity of tissue-specific promoters or elements, as well as assays to characterize their activity, is well known to those of skill in the art. Non-limiting examples of such regions include the human LIMK2 gene (Nomoto et al. 1999), the somatostatin receptor 2 gene (Kraus et al., 1998), murine epididymal retinoic acid-binding gene (Lareyre et al., 1999), human CD4 (Zhao-Emonet et al., 1998), mouse alpha2 (XI) collagen (Tsumaki, et al., 1998), D1A dopamine receptor gene (Lee, et al., 1997), insulin-like growth factor II (Wu et al., 1997), and human platelet endothelial cell adhesion molecule-1 (Almendro et al., 1996).

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

The use of internal ribosome entry sites (IRES) elements may be used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, each herein incorporated by reference).

Most transcribed eukaryotic RNA molecules will undergo RNA splicing to remove introns from the primary transcripts. Vectors containing genomic eukaryotic sequences may require donor and/or acceptor splicing sites to ensure proper processing of the transcript for protein expression (see, for example, Chandler et al., 1997, herein incorporated by reference.)

The vectors or constructs of the present invention will generally comprise at least one termination signal. A “termination signal” or “terminator” is comprised of the DNA sequences involved in specific termination of an RNA transcript by an RNA polymerase. Thus, in certain embodiments a termination signal that ends the production of an RNA transcript is contemplated. A terminator may be necessary in vivo to achieve desirable message levels.

In eukaryotic systems, the terminator region may also comprise specific DNA sequences that permit site-specific cleavage of the new transcript so as to expose a polyadenylation site. This signals a specialized endogenous polymerase to add a stretch of about 200 A residues (polyA) to the 3′ end of the transcript. RNA molecules modified with this polyA tail appear to be more stable and are translated more efficiently. Thus, in other embodiments involving eukaryotes, it is preferred that that terminator comprises a signal for the cleavage of the RNA, and it is more preferred that the terminator signal promotes polyadenylation of the message. The terminator and/or polyadenylation site elements can serve to enhance message levels and to minimize read through from the cassette into other sequences.

Terminators contemplated for use in the invention include any known terminator of transcription described herein or known to one of ordinary skill in the art, including but not limited to, for example, the termination sequences of genes, such as for example the bovine growth hormone terminator or viral termination sequences, such as for example the SV40 terminator. In certain embodiments, the termination signal may be a lack of transcribable or translatable sequence, such as due to a sequence truncation.

In expression, particularly eukaryotic expression, one will typically include a polyadenylation signal to effect proper polyadenylation of the transcript. The nature of the polyadenylation signal is not believed to be crucial to the successful practice of the invention, and any such sequence may be employed. Preferred embodiments include the SV40 polyadenylation signal or the bovine growth hormone polyadenylation signal, convenient and known to function well in various target cells. Polyadenylation may increase the stability of the transcript or may facilitate cytoplasmic transport.

In order to propagate a vector in a host cell, it may contain one or more origins of replication sites (often termed “ori”), which is a specific nucleic acid sequence at which replication is initiated. Alternatively an autonomously replicating sequence (ARS) can be employed if the host cell is yeast.

In certain embodiments of the invention, cells containing a nucleic acid construct of the present invention may be identified in vitro or in vivo by including a marker in the expression vector. Such markers would confer an identifiable change to the cell permitting easy identification of cells containing the expression vector.

Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selectable markers. In addition to markers conferring a phenotype that allows for the discrimination of transformants based on the implementation of conditions, other types of markers including screenable markers such as GFP, whose basis is calorimetric analysis, are also contemplated. Alternatively, screenable enzymes such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selectable and screenable markers are well known.

Kits

The present invention additionally comprises a kit comprising an immunogenic composition, modified cancer cell or pharmaceutical composition of the invention as described herein.

In the case of a kit for therapeutic/prophylactic use, the kit can additionally comprise a pharmaceutically acceptable carrier.

Optionally a kit of the invention is packaged with instructions for use in a method or use of the invention as described herein.

### EXAMPLES

### Example 1

### Identification of Novel Adjuvants Which Enhance CD8+ T Cell Expansion

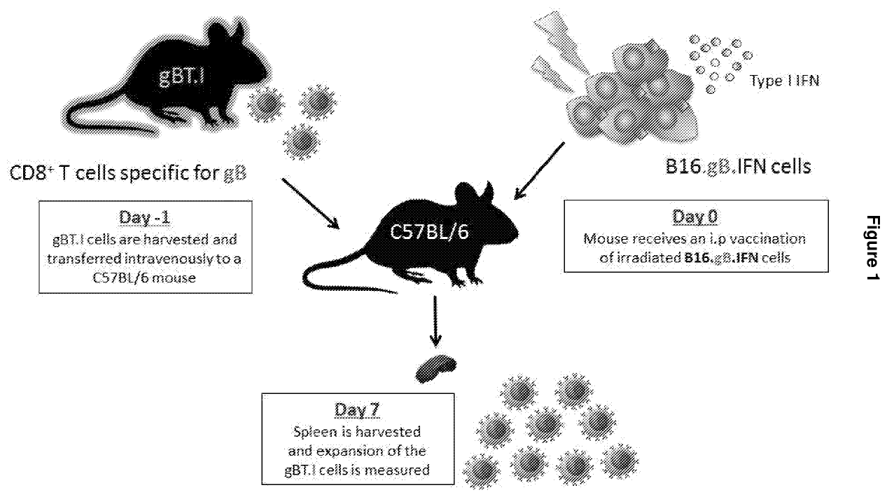

A series of experiments using preclinical models were conducted to test the adjuvant activities of seven members of the type I interferon family in the context of anti-tumour vaccination for melanoma. These models involved the adoptive transfer of a low precursor frequency (5×10⁴) of gBT.I.CD45.1+ CD8+ T cells intravenously into mice at least one day prior to intraperitoneal immunisation with 2.5×10⁶ transduced irradiated B16.gB.GFP cells (±poly I:C or IFNα/β). Seven days post vaccination, the spleen was harvested and the expansion of gBT.I.CD45.1 CD8+ T cells was measured (FIG. 1).

gBT.I: The CD8+ T cells in this TCR transgenic animal express a H-2K^{b}-restricted T cell receptor specific for herpes simplex virus glyocoprotein B (gB). (Mueller SN, Immunol Cell Biology, 2002).

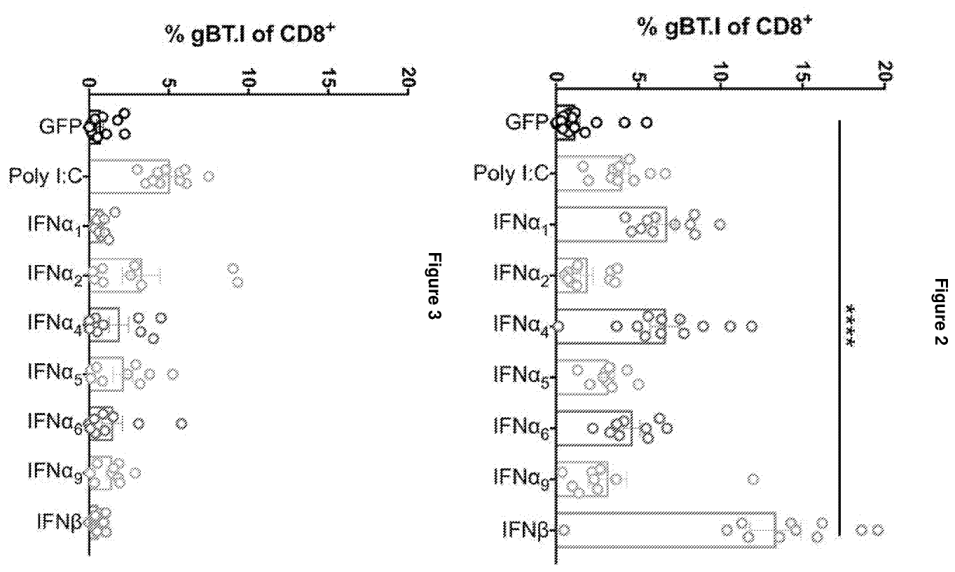

Three of the interferon subtypes induced greater tumour-specific CD8+ T cell expansion than the standard poly I:C adjuvant; this effect was most striking for IFN-β, which showed adjuvant activity around 3 times higher than poly I:C (FIG. 2). When similar experiments were repeated with mice lacking the receptor through which all type I interferons signal, IFNAR^{o/o} mice (Muller U, Science, 1994), the results indicated that signalling though IFNAR on host cells is required for IFN-β to have full adjuvant activity in the experimental model (FIG. 3).

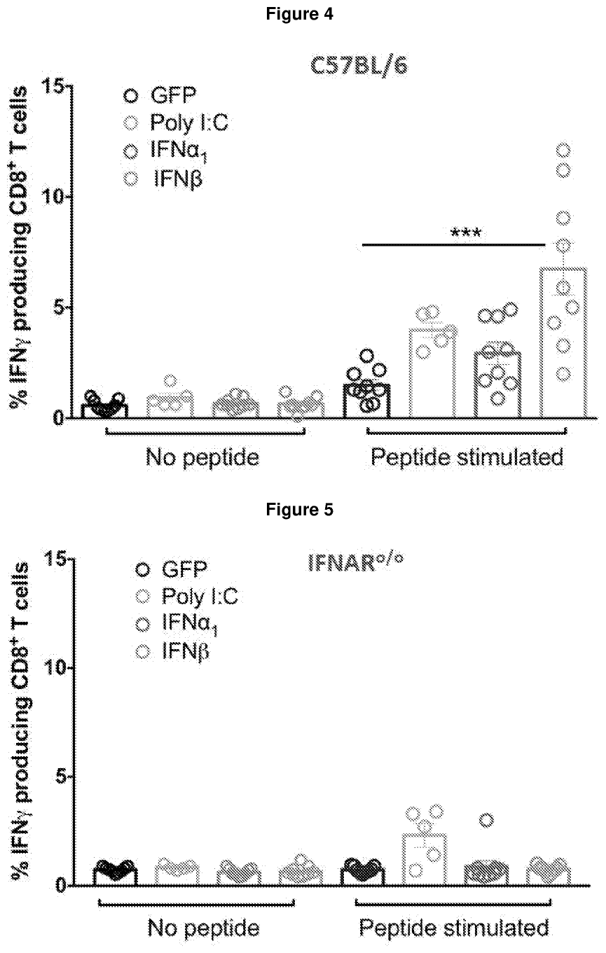

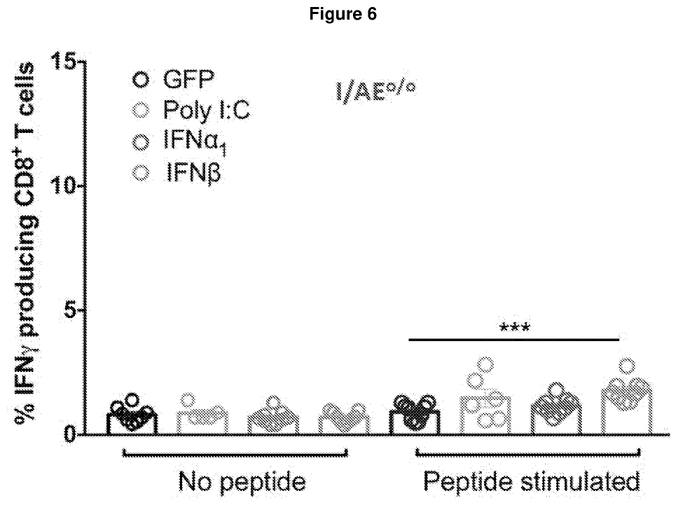

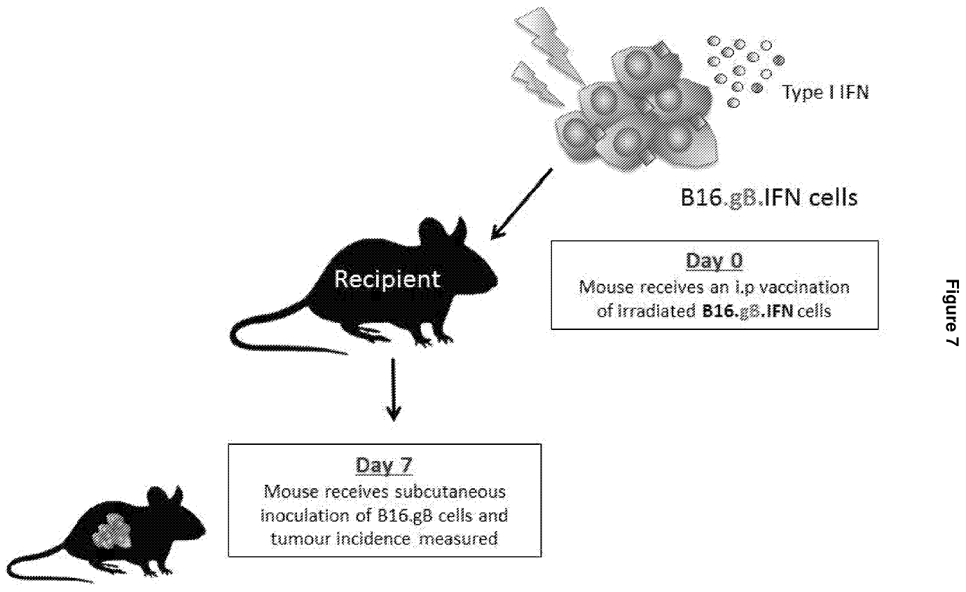

It was next examined whether the enhanced recruitment of gBT.I CD8⁺ T cell activity mediated by IFN-β during vaccination could be repeated in the host T cell compartment. The experiments detailed above were repeated using the strongest type I interferon candidates, this time measuring the expansion of endogenous tumour-specific CD8+ T cells by standard intracellular cytokine staining for IFNγ (Pang K C, J Immunol, 2006). Consistent with previous results, endogenous tumour-specific CD8⁺ T cell expansion was significantly enhanced by IFN-β (FIG. 4) and was dependent on intact IFNAR signalling (FIG. 5). Furthermore, experiments performed in I/AE^{o/o} mice that lack CD4+ T cells demonstrate tumour-specific CD8+ T cell expansion was reduced (FIG. 6), implying that CD4+ help is required for optimal expansion during vaccination with IFN-β.

### Example 2

### Prophylactic Vaccination in Conjunction with Agonism of Interferon Beta Receptor

The efficacy of prophylactic vaccination with IFN-β was further assessed using a subcutaneous B16 melanoma model engineered to express gB from herpes simplex virus. At Day 0, mice receive an i.p. vaccination with 2.5×10⁵ irradiated B16.gB cells expressing GFP±IFNα₁/β. At Day 7, C57BL/6 mice received subcutaneous inoculation of 5×10⁵ B16.gB cells and tumour-free survival is measured (FIG. 7).

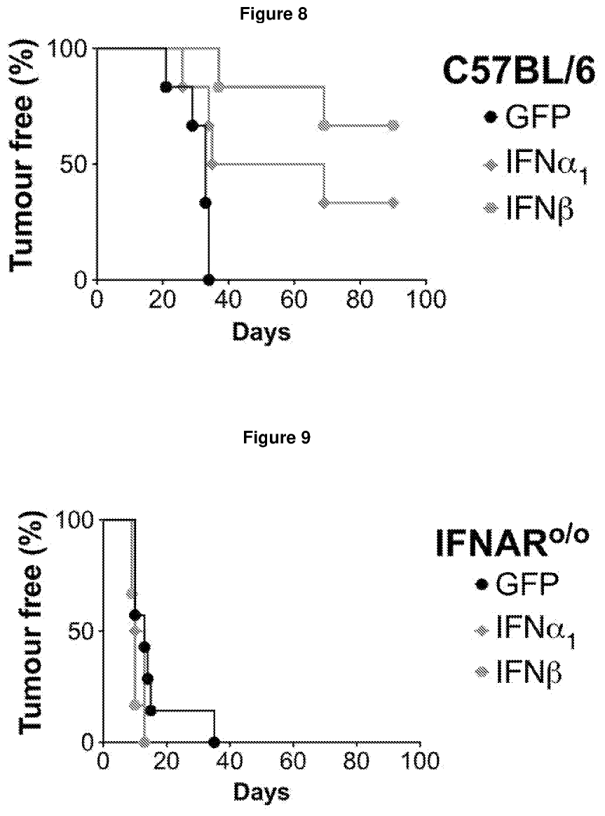

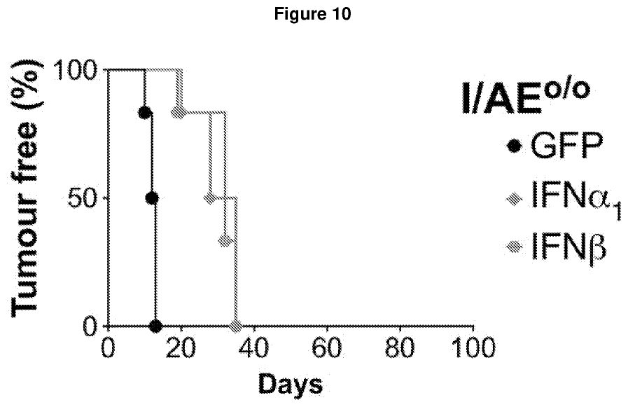

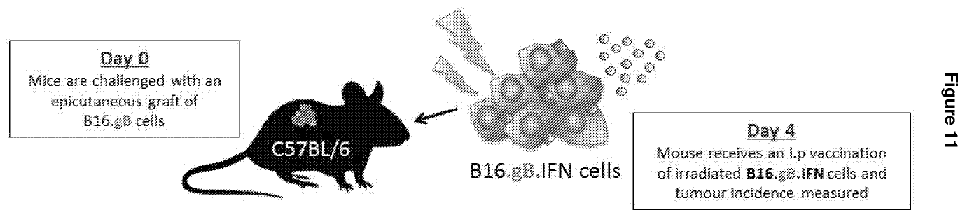

The results demonstrate vaccination with IFN-β results in increased incidence of tumour-free survival, whereas all mice developed large tumours post vaccination in the absence of an interferon subtype, and a reduced proportion of mice vaccinated with IFNα₁ remained tumour-free (FIG. 8). No protection was observed in IFNAR^{o/o} mice receiving vaccination±IFNα₁/β (FIG. 9), demonstrating host IFNα/β signalling is required for the observed protection in C57BL/6 mice. Furthermore, no protection was observed in I/AE^{o/o} mice in these experiments, demonstrating CD4+ T cells are required for the efficacy provided by vaccination with IFN-β (FIG. 10).

### Example 3

### Therapeutic Vaccination in Conjunction with Agonism of Interferon Beta Receptor

The efficacy of therapeutic vaccination with IFN-β was next assessed using a cutaneous B16 melanoma model (Wylie B, Oncoimmunology, 2015) engineered to express gB from herpes simplex virus. At Day 0, C57BL/6 mice received an epicutaneous graft of 10⁵ B16.gB cells. Four days after tumour engraftment, mice were immunised i.p. with either saline or 2×10⁵ irradiated B16.gB cells in the absence (GFP) or presence of IFN-β and tumour incidence measured (FIG. 11).

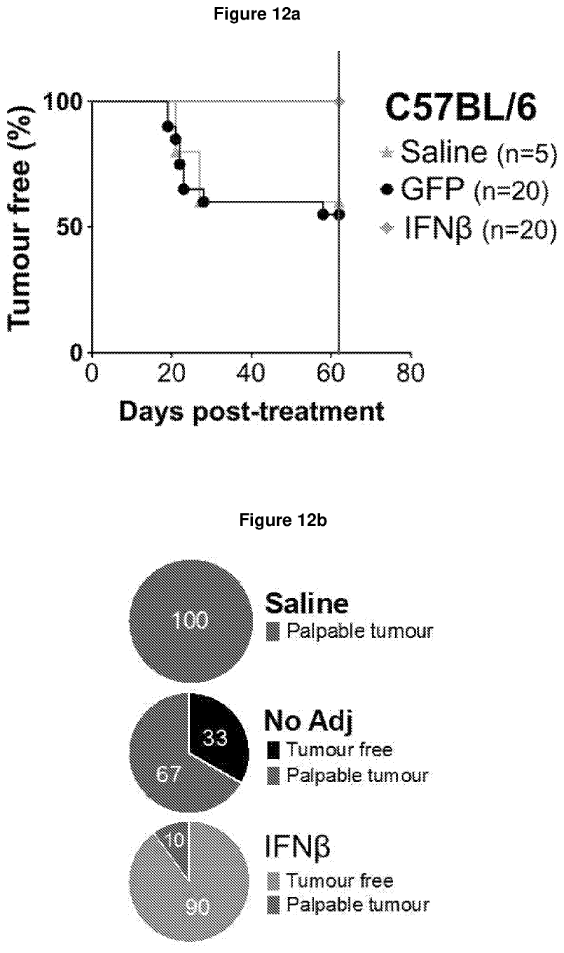

In saline treated or B16-gB vaccinated mice, 40% and 45% of mice respectively developed tumours 60 days after treatment (FIG. 12A), whereas 0% of mice vaccinated with B16-gB cells expressing IFNβ developed tumours (FIG. 12A). Mice surviving 60 days post treatment were re-challenged subcutaneously with B16.gB cells (10⁵) to assess whether memory cells can provide protection. In B16.gB.GFP or B16.gB.GFP+IFNβ vaccinated mice, 67% and 90% of mice respectively, were tumour free 60 days post re-challenge (FIG. 12B). Saline-treated mice were not protected, and all mice developed large tumours within 2-3 weeks (FIG. 12B). These results demonstrate that the vaccination strategy is effective against an established non-immunogenic tumour and is capable of providing protective memory responses.

### Example 4

### Cross-Presenting XCR1+ DCs are essential for CD8+ T cell Expansion

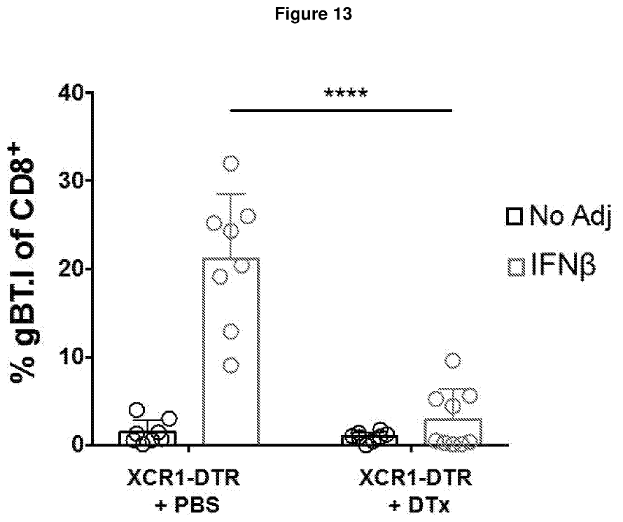

Transgenic XCR1-DTR mice express a primate diphtheria toxin (DTx) receptor (DTR), allowing for conditional depletion of cross-presenting XCR1+ DCs in the presence of DTx. The expansion of tumour-specific CD8+ T cells was measured in vaccinated XCR1-DTR mice treated with either saline (PBS) or DTx. Results are shown in FIG. 13.

### Example 5

### Therapeutic Vaccination, Agonism of Interferon Beta Receptor in Combination with PDL1 Checkpoint Blockade

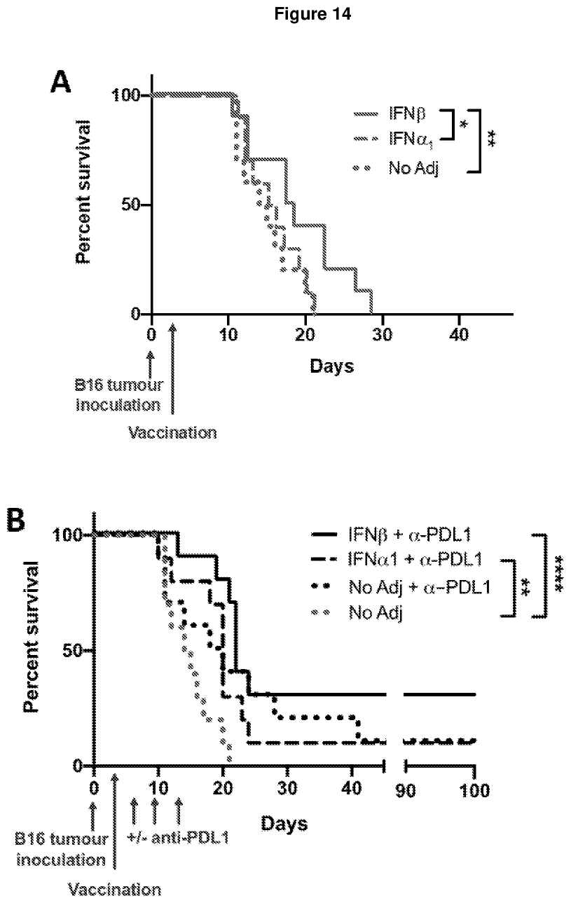

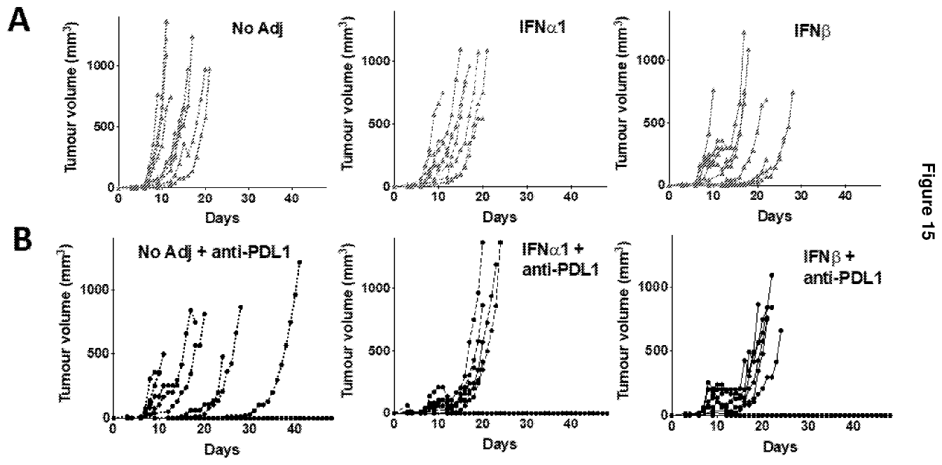

FIGS. 14 and 15 show the results of combining IFNβ expression with anti-PD-L1 blockade therapy. Mice were challenged with 5×10⁵ B16.gB cells subcutaneously. Mice received (A) vaccination alone with 2.5×10⁶ irradiated B16.Kbloss.gB.GFP±IFNα1 OR IFNβ (n=10 per group) i.p. three days post-tumour inoculation or (B) vaccination plus three doses of 200 μg anti-PDL1 (clone 10F.9G2, Biolegend) (n=10 per group) i.p. on days 6, 9 and 12 post-tumour inoculation.

FIG. 14A demonstrates the superiority of IFNβ therapy compared to no adjuvant or IFNα adjuvant therapy, with statistically significant increased percentage survival observed for mice receiving IFNβ compared to no adjuvant or IFNα.

The results shown in FIG. 14B demonstrate that vaccination with IFNβ also synergises with anti-PDL1 checkpoint blockade therapy to delay tumour progression. Statistical significance was determined by Log-rank Mantel-Cox test where *p<0.1, **p<0.05, ****p<0.001.

In particular, a significant proportion of mice receiving IFNβ in combination with anti-PDL1 checkpoint blockade therapy recovered fully from their tumours and were tumour-free beyond 100 days. The results from the combination of IFNβ and anti-PDL1 checkpoint blockade therapy were superior to anti-PDL1 checkpoint blockade therapy alone, and to IFNβ treatment alone (* p<0.1).

FIG. 15 also demonstrates that the combination of IFNβ with anti-PDL1 checkpoint blockade therapy leads to a significant reduction in tumour volume (as well as reduced tumour burden) in challenged mice. FIG. 15 shows individual tumour growth of mice challenged with 5×10⁵ B16.gB cells subcutaneously. Mice received the indicated vaccination (A) alone or (B) in combination with three doses of anti-PDL1 (n=10 per group).

It will be understood that the invention disclosed and defined in this specification extends to all alternative combinations of two or more of the individual features mentioned or evident from the text or drawings. All of these different combinations constitute various alternative aspects of the invention.

1. An immunogenic composition comprising, consisting essentially of or consisting of:
a) a cell expressing an interferon-β (IFN-β) receptor agonist, and
b) a tumour antigen.

2. The immunogenic composition of claim 1, wherein the IFN-β receptor agonist is secreted from the cell, and/or presented on the outer membrane of the cell.

3. The immunogenic composition of claim 1 or 2, wherein the tumour antigen is provided in or on a cell.

4. The immunogenic composition of claim 3, wherein the cell comprising the tumour antigen is a modified human cancer cell.

5. The immunogenic composition of any one of the preceding claims, wherein the composition comprises, consists of or consists essentially of a cell that has been genetically modified to express the tumour antigen and the IFN-β receptor agonist.

6. The immunogenic composition of any one of the preceding claims, wherein the tumour antigen the tumour antigen is a protein, or a fragment, peptide or derivative thereof, produced by a tumour cell, preferably, wherein the tumour antigen is a tumour-specific antigen.

7. The immunogenic composition of any one of the preceding claims wherein the cell expressing an IFN-β receptor agonist is a T cell, natural killer (NK) cell or an antigen presenting cell.

8. The immunogenic composition of claim 7 wherein the cell expressing the IFN-β receptor agonist is a dendritic cell, macrophage, or a B cell.

9. The immunogenic composition of claim 7 or 8, wherein the cell also expresses the tumour antigen.

10. The immunogenic composition of any one of claims 1 to 6, wherein the cell expressing an IFN-β receptor agonist is a human cancer cell that has been modified to express the IFN-β receptor agonist.

11. The immunogenic composition of any one of the preceding claims wherein the IFN-β receptor agonist is the IFN-β polypeptide, or an immunologically active fragment, variant or derivative thereof.

12. The immunogenic composition of any one of the preceding claims wherein the IFN-β receptor agonist is an IFN-β mimetic.

13. The immunogenic composition of any one of the preceding claims wherein the IFN-β receptor agonist is an antibody that binds to the IFN-β receptor and agonises the receptor.

14. A modified cancer cell, wherein the cell has been modified to express an IFN-β receptor agonist.

15. The modified cancer cell of claim 14 wherein IFN-β receptor agonist is the IFN-β polypeptide, an immunologically active fragment, variant or derivative thereof, or an IFN-β mimetic.

16. The modified cancer cell of claim 14 or 15, wherein the cell has been inactivated or irradiated to prevent the cell from forming a tumour.

17. A pharmaceutical composition for treating or preventing cancer in an individual comprising, consisting essentially of or consisting of:
a) a cell expressing an interferon-β (IFN-β) receptor agonist,
b) a tumour antigen,
and a pharmaceutically acceptable diluent, excipient or carrier.

18. The pharmaceutical composition of claim 17, wherein the active ingredient in the composition is the cell expressing an interferon-β (IFN-β) receptor agonist and the tumour antigen.

19. The pharmaceutical composition of claim 17, wherein the main ingredient in the composition is the cell expressing an interferon-β (IFN-β) receptor agonist and the tumour antigen.

20. The pharmaceutical composition of any one of claims 17 to 19, wherein the pharmaceutical composition comprises, consists essentially of or consists of a) a cell expressing an interferon-β (IFN-β) receptor agonist and b) a tumour antigen.

21. The pharmaceutical composition of any one of claims 17 to 20, wherein the cell is a modified human cancer cell expressing an IFN-β receptor agonist.

22. The pharmaceutical composition of any one of claims 17 to 20, wherein the cell is a T cell, NK cell or antigen presenting cell which expresses an IFN-β receptor agonist.

23. The pharmaceutical composition of any one of claims 17 to 20, wherein the pharmaceutical composition comprises, consists essentially of, or consists of a cell expressing an IFN-β receptor agonist, and a cell expressing a tumour antigen, such as a modified human cancer cell that has been modified to prevent the cell from forming a tumour.

24. A method of treating cancer comprising administering an immunogenic composition of any one of claims 1 to 13, a modified cancer cell of any one of claims 14 to 16, or pharmaceutical composition of any one of claims 17 to 23 to an individual in need thereof, thereby treating the cancer.

25. A method of treating cancer comprising
providing an individual in need of cancer treatment; and
administering an immunogenic composition of any one of claims 1 to 13, a modified cancer cell of claim 14 or 16 or a pharmaceutical composition of any one of claims 16 to 23 to the individual,
thereby treating the cancer.

26. Use of an immunogenic composition of any one of claims 1 to 13, or a modified cancer cell of any one of claims 14 to 16 in the manufacture of a medicament for the treatment or prevention of cancer in an individual.

27. An immunogenic composition of any one of claims 1 to 13, a modified cancer cell of any one of claims 14 to 16 or a pharmaceutical composition of any one of claims 17 to 23 for use in the treatment or prevention of cancer in an individual.

28. A method for inducing an immune response suitable for the treatment of cancer in an individual, the method comprising administering an immunogenic composition of any one of claims 1 to 13, a modified cancer cell of any one of claims 14 to 16 or a pharmaceutical composition of any one of claims 17 to 23, to an individual in need thereof, thereby inducing an immune response and thereby treating the cancer.

29. A method of inducing a T cell immune response in an individual, the method comprising administering an immunogenic composition of any one of claims 1 to 13, a modified cancer cell of any one of claims 14 to 16 or a pharmaceutical composition of any one of claims 17 to 23, to an individual in need thereof, thereby inducing a T cell immune response,
wherein preferably the T cell immune response is a cytotoxic T cell immune response.

30. A method of treating cancer in an individual, the method comprising
obtaining a cancer cell from an individual in need of treatment for cancer,
modifying the cancer cell to (a) reduce or remove the ability of the cell to form a tumour and (b) express an IFN-β receptor agonist, and
administering the modified cancer cell to the individual,
thereby treating cancer in the individual.

31. A method for producing a modified cancer cell, the method comprising
providing a cancer cell,
inactivating the cancer cell to reduce or remove the ability of the cell to form a tumour, and
modifying the inactivated cancer cell to express an IFNβ receptor agonist, thereby producing a modified cancer cell.

32. The method of any one of claim 30 or 31 wherein the IFN-β receptor agonist is an IFN-β polypeptide, an immunologically active fragment, variant or derivative thereof; or an IFN-β mimetic.

33. The method of any one of claims 25 or 30 to 32 wherein the method further comprises administration of a checkpoint inhibitor to the individual.

34. The method of claim 33, wherein the checkpoint inhibitor is selected from an inhibitor of CTLA4, PD-1, and/or PD-L1.

35. The method of claim 34, wherein the checkpoint inhibitor is a PD-L1 inhibitor.

36. The immunogenic composition of claim 4, or the modified cancer cell of any one of claim 14 or 16, wherein the cancer cell is an autologous cancer cell from the subject being treated.

37. The immunogenic composition of claim 4, or the modified cancer cell of any one of claim 14 or 16, wherein the cancer cell is an allogenic cancer cell.

38. The immunogenic composition of any one of claims 1 to 13, the modified cancer cell of any one of claims 14 to 16, or the pharmaceutical composition of any one of claims 17 to 23, wherein the compositions or cells are administered to a subject requiring treatment, as a vaccine.
